(19) United States
(12) Patent Application Publication
(10) Pub. No.: US 2023/0282367 A1
(43) Pub. Date: Sep. 7, 2023

(54) METHODS AND SYSTEMS FOR PREDICTING RESPONSE TO ANTI-TNF THERAPIES

(71) Applicant: Scipher Medicine Corporation, Waltham, MA (US)

(72) Inventors: Susan GHIASSIAN, Boston, MA (US); Marc SANTOLINI, Waltham, MA (US); Nancy SCHOENBRUNNER, Charlestown, MA (US); Keith J. JOHNSON, Wayland, MA (US)

(21) Appl. No.: 18/176,288

(22) Filed: Feb. 28, 2023

Related U.S. Application Data
(63) Continuation of application No. PCT/US2021/048346, filed on Aug. 31, 2021.
(60) Provisional application No. 63/073,336, filed on Sep. 1, 2020.

(51) Int. Cl. G16H 50/70; G16B 20/00; G16B 40/20

(52) U.S. Cl. CPC G16H 50/70; G16B 20/00; G16B 40/20

(57) ABSTRACT

Methods and systems for administering therapy to subjects who have been determined to not display a gene expression response signature established to distinguish between responsive and non-responsive prior subjects who have received anti-TNF therapy.

### CROSS-REFERENCE TO RELATED APPLICATIONS

The present application is a continuation of International Application No. PCT/US2021/048346, filed Aug. 31, 2021, which claims priority to U.S. Provisional Application No. 63/073,336, filed Sep. 1, 2020, the content of each which is incorporated by reference herein in its entirety.

### BACKGROUND

Tumor necrosis factor (TNF) is a cell signaling protein related to regulation of immune cells and apoptosis and is implicated in a variety of immune and autoimmune-mediated disorders. In particular, TNF is known to promote inflammatory response, which causes many problems associated with autoimmune disorders, such as rheumatoid arthritis, psoriatic arthritis, ankylosing spondylitis, Crohn's disease, ulcerative colitis, inflammatory bowel disease, chronic psoriasis, hidradenitis suppurativa, asthma, juvenile idiopathic arthritis, vitiligo, Graves' ophthalmopathy (also known as thyroid eye disease, or Graves' orbitopathy), and multiple sclerosis.

TNF-mediated disorders are currently treated by inhibition of TNF, and in particular by administration of an anti-TNF agent (i.e., by anti-TNF therapy). Examples of anti-TNF agents approved in the United States include monoclonal antibodies that target TNF, such as adalimumab (Humira®), certolizumab pegol (Cimiza®), golimumab (Simponi® and Simponi Aria®), and infliximab (Remicade®), decoy circulating receptor fusion proteins such as etanercept (Enbrel®), and biosimilars, such as adalimumab ABP 501 (AMGEVITA™), and etanercept biosimilars GP2015 (Erelzi®).

### SUMMARY

A significant known problem with anti-TNF therapies is that response rates are inconsistent. Indeed, recent international conferences designed to bring together leading scientists and clinicians in the fields of immunology and rheumatology to identify unmet needs in these fields almost universally identify uncertainty in response rates as an ongoing challenge. For example, the 19^{th} annual International Targeted Therapies meeting, which held break-out sessions relating to challenges in treatment of a variety of diseases, including rheumatoid arthritis, psoriatic arthritis, axial spondyloarthritis, systemic lupus erythematous, and connective tissue diseases (e.g. Sjogren's syndrome, Systemic sclerosis, vasculitis including Bechet's and IgG4 related disease), identified certain issues common to all of these diseases, specifically, “the need for better understanding the heterogeneity within each disease . . . so that predictive tools for therapeutic responses can be developed. See Winthrop, et al., “The unmet need in rheumatology: Reports from the targeted therapies meeting 2017,” Clin. Immunol. pii: S1521-6616(17)30543-0, Aug. 12, 2017. Similarly, extensive literature relating to treatment of Crohn's Disease with anti-TNF therapy consistently bemoans erratic response rates and inability to predict which patients will benefit. See, e.g., M. T. Abreu, “Anti-TNF Failures in Crohn's Disease,” Gastroenterol Hepatol (N.Y.), 7(1):37-39 (Jan. 2011); see also Ding et al., “Systematic review: predicting and optimising response to anti-TNF therapy in Crohn's disease—algorithm for practical management,” Aliment Pharmacol. Ther., 43(1):30-51 (Jan. 2016) (reporting that “[p]rimary nonresponse to anti-TNF treatment affects 13-40% of patients.”).

Thus, a significant number of patients to whom anti-TNF therapy is currently being administered do not benefit from the treatment, and could even be harmed. Known risks of serious infection and malignancy associated with anti-TNF therapy are so significant that product approvals typically require so-called “black box warnings” be included on the label. Other potential side effects of such therapy include, for example, congestive heart failure, demyelinating disease, and other systemic side effects. Furthermore, given that several weeks to months of treatment are required before a patient is identified as not responding to anti-TNF therapy (i.e., is a non-responder to anti-TNF therapy), proper treatment of such patients can be significantly delayed as a result of the current inability to identify responder vs non-responder subjects. See, e.g., Roda et al., “Loss of Response to Anti-TNFs: Definition, Epidemiology, and Management,” Clin. Tranl. Gastroenterol., 7(1):e135 (January 2016) (citing Hanauer et al., “ACCENT I Study group. Maintenance Infliximab for Crohn's disease: the ACCENT I randomized trial,” Lancet 59:1541-1549 (2002); Sands et al., “Infliximab maintenance therapy for fistulizing Crohn's disease,” N. Engl. J. Med. 350:876-885 (2004)).

Taken together, particularly given that these anti-TNF therapies can be quite expensive (typically costing upwards of $40,000-60,000 per patient per year), these challenges make clear that technologies capable of defining, identifying, and/or characterizing responder vs. non-responder patient populations would represent a significant technological advance, and would provide significant value to patients and to the healthcare industry more broadly, including to doctors, regulatory agencies, and drug developers. The present disclosure provides such technologies.

Provided technologies, among other things, permit care providers to distinguish subjects likely to benefit from anti-TNF therapy from those who are not, reduce risks to patients, increase timing and quality of care for non-responder patient populations, increase efficiency of drug development, and avoid costs associated with administering ineffective therapy to non-responder patients or with treating side effects such patients experience upon receiving anti-TNF therapy.

Provided technologies embody and/or arise from, among other things, certain insights that include, for example, identification of the source of a problem with certain conventional approaches to defining responder vs. non-responder populations and/or that represent particularly useful strategies for defining classifiers that distinguish between such populations. For example, as described herein, the present disclosure identifies that one source of a problem with many conventional strategies for defining responder vs. non-responder populations through consideration of gene expression differences in the populations is that they typically prioritize or otherwise focus on highest fold changes; the present disclosure teaches that such an approach misses subtle but meaningful differences relevant to disease biology. Moreover, the present disclosure offers an insight that mapping of genes with altered expression levels onto a human interactome map (in particular onto a human interactome map that represents experimentally supported physical interactions between cellular components which, in some embodiments, explicitly excludes any theoretical, calculated, or other interaction that has been proposed but not experimentally validated), can provide a useful and effective classifier for defining responders vs. non-responders to anti-TNF therapy. In some embodiments, genes included in such a classifier represent a connected module on the human interactome.

In some embodiments, the present disclosure provides a method of treating subjects suffering from a disease, disorder, or condition (e.g., inflammatory bowel disease, ulcerative colitis or Crohn's disease) with anti-TNF therapy, the method comprising a step of: administering the anti-TNF therapy to subjects who have been determined to display a gene expression response signature established to distinguish between responsive and non-responsive prior subjects who have received the anti-TNF therapy (e.g., where “prior subjects” refer to subjects who have previously received the anti-TNF therapy, and have been classified as responsive or non-responsive to said anti-TNF therapy).

In some embodiments, the present disclosure provides method of treating a subject suffering from a disease, disorder, or condition (e.g., inflammatory bowel disease, ulcerative colitis or Crohn's disease) with an anti-TNF therapy, the method comprising a step of: administering the anti-TNF therapy to subjects who have been determined not to display a gene expression response signature established to distinguish between responsive and non-responsive prior subjects who have received the anti-TNF therapy.

In some embodiments, the present disclosure provides method of treating a subject suffering from a disease, disorder, or condition with an anti-TNF therapy, the method comprising a step of: administering the anti-TNF therapy to subjects who have been determined to be responsive via a classifier determined to distinguish between responsive and non-responsive subjects who have received the anti-TNF therapy (“prior subjects”), wherein the classifier distinguishes between responsive and non-responsive subjects on the basis of a set of variables, the set of variables comprising expression of one or more genes selected from: PKM, ADAR, ANP32B, ATRX, BRD7, CAPN1, CCDC88A, CFAP206, CGN, CIRBP, CLTC, EEA1, ERICH1, FAM192A, FAM207A, HHEX, KLF3, LCA5, MDC1, MDM2, NFAT5, ARCN1, ARF6, ARNT, ARPC5L, ASB16, ATF7IP, ATP6VOC, BRF1, CHFR, EDA, EFEMP2, ESR2, FAM179B, FTH1, H3F3A, HDAC4, HINFP, HNRNPK, SUMO2, NUCKS1, PML, PNN, PRKAB1, RBCK1, RRP15, SNRPN, TFIP11, THTPA, TMEM87A, TNK2, TPR, TRAPPC4, UBA5, UBE2D1, VPS72, YWHAE, MCM5, MED6, MGST2, MSH6, PURA, RABGEF1, RBBP6, RBM26, RECQL, RUNX3, SFPQ, SGCB, SMARCA1, SMC1A, SPAG9, UBA2, UBE2B, USPL1, HP1BP3, HRAS, or MAX.

In some embodiments, the present disclosure provides a kit for evaluating a likelihood that a subject suffering from an autoimmune disorder will not respond to an anti-TNF therapy, the kit comprising a set of reagents for detecting an expression level of one or more genes selected from the group consisting of: PKM, ADAR, ANP32B, ATRX, BRD7, CAPN1, CCDC88A, CFAP206, CGN, CIRBP, CLTC, EEA1, ERICH1, FAM192A, FAM207A, HHEX, KLF3, LCA5, MDC1, MDM2, NFAT5, ARCN1, ARF6, ARNT, ARPC5L, ASB16, ATF7IP, ATP6VOC, BRF1, CHFR, EDA, EFEMP2, ESR2, FAM179B, FTH1, H3F3A, HDAC4, HINFP, HNRNPK, SUMO2, NUCKS1, PML, PNN, PRKAB1, RBCK1, RRP15, SNRPN, TFIP11, THTPA, TMEM87A, TNK2, TPR, TRAPPC4, UBA5, UBE2D1, VPS72, YWHAE, MCM5, MED6, MGST2, MSH6, PURA, RABGEF1, RBBP6, RBM26, RECQL, RUNX3, SFPQ, SGCB, SMARCA1, SMC1A, SPAG9, UBA2, UBE2B, USPL1, HP1BP3, HRAS, or MAX.

### Definitions

Administration: As used herein, the term “administration” typically refers to the administration of a composition to a subject or system, for example to achieve delivery of an agent that is, or is included in or otherwise delivered by, the composition.

Agent: As used herein, the term “agent” refers to an entity (e.g., for example, a lipid, metal, nucleic acid, polypeptide, polysaccharide, small molecule, etc., or complex, combination, mixture or system [e.g., cell, tissue, organism] thereof), or phenomenon (e.g., heat, electric current or field, magnetic force or field, etc.).

Amino acid: As used herein, the term “amino acid” refers to any compound and/or substance that can be incorporated into a polypeptide chain, e.g., through formation of one or more peptide bonds. In some embodiments, an amino acid has the general structure H₂N—C(H)(R)—COOH. In some embodiments, an amino acid is a naturally-occurring amino acid. In some embodiments, an amino acid is a non-natural amino acid; in some embodiments, an amino acid is a D-amino acid; in some embodiments, an amino acid is an L-amino acid. As used herein, the term “standard amino acid” refers to any of the twenty L-amino acids commonly found in naturally occurring peptides. “Nonstandard amino acid” refers to any amino acid, other than the standard amino acids, regardless of whether it is or can be found in a natural source. In some embodiments, an amino acid, including a carboxy- and/or amino-terminal amino acid in a polypeptide, can contain a structural modification as compared to the general structure above. For example, in some embodiments, an amino acid may be modified by methylation, amidation, acetylation, pegylation, glycosylation, phosphorylation, and/or substitution (e.g., of the amino group, the carboxylic acid group, one or more protons, and/or the hydroxyl group) as compared to the general structure. In some embodiments, such modification may, for example, alter the stability or the circulating half-life of a polypeptide containing the modified amino acid as compared to one containing an otherwise identical unmodified amino acid. In some embodiments, such modification does not significantly alter a relevant activity of a polypeptide containing the modified amino acid, as compared to one containing an otherwise identical unmodified amino acid. As will be clear from context, in some embodiments, the term “amino acid” may be used to refer to a free amino acid; in some embodiments it may be used to refer to an amino acid residue of a polypeptide, e.g., an amino acid residue within a polypeptide.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Antagonist: As used herein, the term “antagonist” may refer to an agent, or condition whose presence, level, degree, type, or form is associated with a decreased level or activity of a target. An antagonist may include an agent of any chemical class including, for example, small molecules, polypeptides, nucleic acids, carbohydrates, lipids, metals, and/or any other entity that shows the relevant inhibitory activity. In some embodiments, an antagonist may be a “direct antagonist” in that it binds directly to its target; in some embodiments, an antagonist may be an “indirect antagonist” in that it exerts its influence by means other than binding directly to its target; e.g., by interacting with a regulator of the target, so that the level or activity of the target is altered). In some embodiments, an “antagonist” may be referred to as an “inhibitor”.

Antibody: As used herein, the term “antibody” refers to a polypeptide that includes canonical immunoglobulin sequence elements sufficient to confer specific binding to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)— an amino-terminal variable (VH) domain (located at the tips of the Y structure), followed by three constant domains: CH1, CH2, and the carboxy-terminal CH3 (located at the base of the Y's stem). A short region, known as the “switch”, connects the heavy chain variable and constant regions. The “hinge” connects CH2 and CH3 domains to the rest of the antibody. Two disulfide bonds in this hinge region connect the two heavy chain polypeptides to one another in an intact antibody. Each light chain is comprised of two domains—an amino-terminal variable (VL) domain, followed by a carboxy-terminal constant (CL) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the CH2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “complement determining regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity. As is known in the art, affinity and/or other binding attributes of Fc regions for Fc receptors can be modulated through glycosylation or other modification. In some embodiments, antibodies produced and/or utilized in accordance with the present invention include glycosylated Fc domains, including Fc domains with modified or engineered such glycosylation. For purposes of the present invention, in certain embodiments, any polypeptide or complex of polypeptides that includes sufficient immunoglobulin domain sequences as found in natural antibodies can be referred to and/or used as an “antibody”, whether such polypeptide is naturally produced (e.g., generated by an organism reacting to an antigen), or produced by recombinant engineering, chemical synthesis, or other artificial system or methodology. In some embodiments, an antibody is polyclonal; in some embodiments, an antibody is monoclonal. In some embodiments, an antibody has constant region sequences that are characteristic of mouse, rabbit, primate, or human antibodies. In some embodiments, antibody sequence elements are humanized, primatized, chimeric, etc, as is known in the art. Moreover, the term “antibody” as used herein, can refer in appropriate embodiments (unless otherwise stated or clear from context) to any of the art-known or developed constructs or formats for utilizing antibody structural and functional features in alternative presentation. For example, embodiments, an antibody utilized in accordance with the present invention is in a format selected from, but not limited to, intact IgA, IgG, IgE or IgM antibodies; bi- or multi-specific antibodies (e.g., Zybodies®, etc.); antibody fragments such as Fab fragments, Fab′ fragments, F(ab′)2 fragments, Fd′ fragments, Fd fragments, and isolated CDRs or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPs™”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc], or other pendant group [e.g., poly-ethylene glycol, etc.]).

Associated: Two events or entities are “associated” with one another, as that term is used herein, if the presence, level, degree, type and/or form of one is correlated with that of the other. For example, a particular entity (e.g., polypeptide, genetic signature, metabolite, microbe, etc) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

Biological Sample: As used herein, the term “biological sample” typically refers to a sample obtained or derived from a biological source (e.g., a tissue or organism or cell culture) of interest, as described herein. In some embodiments, a source of interest comprises an organism, such as an animal or human. In some embodiments, a biological sample is or comprises biological tissue or fluid. In some embodiments, a biological sample may be or comprise bone marrow; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as a ductal lavages or broncheoalveolar lavages; aspirates; scrapings; bone marrow specimens; tissue biopsy specimens; surgical specimens; feces, other body fluids, secretions, and/or excretions; and/or cells therefrom, etc. In some embodiments, a biological sample is or comprises cells obtained from an individual. In some embodiments, obtained cells are or include cells from an individual from whom the sample is obtained. In some embodiments, a sample is a “primary sample” obtained directly from a source of interest by any appropriate means. For example, in some embodiments, a primary biological sample is obtained by methods selected from the group consisting of biopsy (e.g., fine needle aspiration or tissue biopsy), surgery, collection of body fluid (e.g., blood, lymph, feces etc.), etc. In some embodiments, as will be clear from context, the term “sample” refers to a preparation that is obtained by processing (e.g., by removing one or more components of and/or by adding one or more agents to) a primary sample. For example, filtering using a semi-permeable membrane. Such a “processed sample” may comprise, for example nucleic acids or proteins extracted from a sample or obtained by subjecting a primary sample to techniques such as amplification or reverse transcription of mRNA, isolation and/or purification of certain components, etc.

Biological Network: As used herein, the term “biological network” refers to any network that applies to biological systems, having sub-units (e.g., “nodes”) that are linked into a whole, such as species units linked into a whole web. In some embodiments, a biological network is a protein-protein interaction network (PPI), representing interactions among proteins present in a cell, where proteins are nodes and their interactions are edges. In some embodiments, connections between nodes in a PPI are experimentally verified. In some embodiments, connections between nodes are a combination of experimentally verified a mathematically calculated. In some embodiments, a biological network is a human interactome (a network of experimentally derived interactions that occur in human cells, which includes protein-protein interaction information as well as gene expression and co-expression, cellular co-localization of proteins, genetic information, metabolic and signaling pathways, etc.). In some embodiments, a biological network is a gene regulatory network, a gene co-expression network, a metabolic network, or a signaling network.

Combination Therapy: As used herein, the term “combination therapy” refers to a clinical intervention in which a subject is simultaneously exposed to two or more therapeutic regimens (e.g. two or more therapeutic agents). In some embodiments, the two or more therapeutic regimens may be administered simultaneously. In some embodiments, the two or more therapeutic regimens may be administered sequentially (e.g., a first regimen administered prior to administration of any doses of a second regimen). In some embodiments, the two or more therapeutic regimens are administered in overlapping dosing regimens. In some embodiments, administration of combination therapy may involve administration of one or more therapeutic agents or modalities to a subject receiving the other agent(s) or modality. In some embodiments, combination therapy does not necessarily require that individual agents be administered together in a single composition (or even necessarily at the same time). In some embodiments, two or more therapeutic agents or modalities of a combination therapy are administered to a subject separately, e.g., in separate compositions, via separate administration routes (e.g., one agent orally and another agent intravenously), and/or at different time points. In some embodiments, two or more therapeutic agents may be administered together in a combination composition, or even in a combination compound (e.g., as part of a single chemical complex or covalent entity), via the same administration route, and/or at the same time.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Corresponding to: As used herein, the phrase “corresponding to” refers to a relationship between two entities, events, or phenomena that share sufficient features to be reasonably comparable such that “corresponding” attributes are apparent. For example, in some embodiments, the term may be used in reference to a compound or composition, to designate the position and/or identity of a structural element in the compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure.

Dosing regimen: As used herein, the term “dosing regimen” refers to a set of unit doses (typically more than one) that are administered individually to a subject, typically separated by periods of time. In some embodiments, a given therapeutic agent has a recommended dosing regimen, which may involve one or more doses. In some embodiments, a dosing regimen comprises a plurality of doses each of which is separated in time from other doses. In some embodiments, individual doses are separated from one another by a time period of the same length; in some embodiments, a dosing regimen comprises a plurality of doses and at least two different time periods separating individual doses. In some embodiments, all doses within a dosing regimen are of the same unit dose amount. In some embodiments, different doses within a dosing regimen are of different amounts. In some embodiments, a dosing regimen comprises a first dose in a first dose amount, followed by one or more additional doses in a second dose amount different from the first dose amount. In some embodiments, a dosing regimen comprises a first dose in a first dose amount, followed by one or more additional doses in a second dose amount same as the first dose amount. In some embodiments, a dosing regimen is correlated with a desired or beneficial outcome when administered across a relevant population (i.e., is a therapeutic dosing regimen).

Improved, increased or reduced: As used herein, the terms “improved,” “increased,” or “reduced,”, or grammatically comparable comparative terms thereof, indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.).

Patient or subject: As used herein, the term “patient” or “subject” refers to any organism to which a provided composition is or may be administered, e.g., for experimental, diagnostic, prophylactic, cosmetic, and/or therapeutic purposes. Typical patients or subjects include animals (e.g., mammals such as mice, rats, rabbits, non-human primates, and/or humans). In some embodiments, a patient is a human. In some embodiments, a patient or a subject is suffering from or susceptible to one or more disorders or conditions. In some embodiments, a patient or subject displays one or more symptoms of a disorder or condition. In some embodiments, a patient or subject has been diagnosed with one or more disorders or conditions. In some embodiments, a patient or a subject is receiving or has received certain therapy to diagnose and/or to treat a disease, disorder, or condition.

Pharmaceutical composition: As used herein, the term “pharmaceutical composition” refers to an active agent, formulated together with one or more pharmaceutically acceptable carriers. In some embodiments, the active agent is present in unit dose amounts appropriate for administration in a therapeutic regimen to a relevant subject (e.g., in amounts that have been demonstrated to show a statistically significant probability of achieving a predetermined therapeutic effect when administered), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

Pharmaceutically acceptable: As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Responder As used herein, the term “responder” refers to a subject that displays an improvement in clinical signs and symptoms after receiving anti-TNF therapy for a period of time. Those skilled in the art will understand that the medical community may establish an appropriate period of time for any particular disease or condition, or for any particular patient or patient type. To give but a few examples, in some embodiments, the period of time may be at least 8 weeks. In some embodiments, the period of time may be at least 12 weeks. In some embodiments, the period of time may be 14 weeks.

Non-Responder: As used herein, the term “non-responder” refers to a subject that displays a insufficient improvement in clinical signs and symptoms after receiving anti-TNF therapy for a period of time. Those skilled in the art will understand that the medical community may establish an appropriate period of time for any particular disease or condition, or for any particular patient or patient type. To give but a few examples, in some embodiments, the period of time may be at least 8 weeks. In some embodiments, the period of time may be at least 12 weeks. In some embodiments, the period of time may be 14 weeks.

Reference: As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

Therapeutic agent: As used herein, the phrase “therapeutic agent” in general refers to any agent that elicits a desired pharmacological effect when administered to an organism. In some embodiments, an agent is considered to be a therapeutic agent if it demonstrates a statistically significant effect across an appropriate population. In some embodiments, the appropriate population may be a population of model organisms. In some embodiments, an appropriate population may be defined by various criteria, such as a certain age group, gender, genetic background, preexisting clinical conditions, etc. In some embodiments, a therapeutic agent is a substance that can be used to alleviate, ameliorate, relieve, inhibit, prevent, delay onset of, reduce severity of, and/or reduce incidence of one or more symptoms or features of a disease, disorder, and/or condition. In some embodiments, a “therapeutic agent” is an agent that has been or is required to be approved by a government agency before it can be marketed for administration to humans. In some embodiments, a “therapeutic agent” is an agent for which a medical prescription is required for administration to humans.

Therapeutically effective amount: As used herein, the term “therapeutically effective amount” refers to an amount of a substance (e.g., a therapeutic agent, composition, and/or formulation) that elicits a desired biological response when administered as part of a therapeutic regimen. In some embodiments, a therapeutically effective amount of a substance is an amount that is sufficient, when administered to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder and/or condition. In some embodiments, a therapeutically effective amount is administered in a single dose; in some embodiments, multiple unit doses are required to deliver a therapeutically effective amount.

Treat: As used herein, the terms “treat,” “treatment,” or “treating” refer to any method used to partially or completely alleviate, ameliorate, relieve, inhibit, prevent, delay onset of, reduce severity of, and/or reduce incidence of one or more symptoms or features of a disease, disorder, and/or condition. Treatment may be administered to a subject who does not exhibit signs of a disease, disorder, and/or condition. In some embodiments, treatment may be administered to a subject who exhibits only early signs of the disease, disorder, and/or condition, for example, for the purpose of decreasing the risk of developing pathology associated with the disease, disorder, and/or condition.

### BRIEF DESCRIPTION OF THE DRAWINGS

FIGS. 1A-1B are plots illustrating ulcerative colitis (UC) response signature genes modules detected using the human interactome (HI) from the UC cohort. The response signature genes found in gene expression data form a significant cluster when mapped to the HI (FIG. 1A) and is much larger than expected by chance (FIG. 1B) which reflects an underlying biology of response.

FIGS. 2A-2B are plots illustrating in-cohort performance of response predictions of a near perfect classifier using leave-one-out cross-validation. FIG. 2A is a receiver operating characteristic (ROC) curve and FIG. 2B illustrates the Negative Predictive Value (NPV) vs. True Negative Rate (TNR) curve. The classifier is able to detect 70% of the non-responders with 100% accuracy, and 100% of the non-responders with 90% accuracy.

FIGS. 3A-3B are plots illustrating cross-cohort performance of response prediction classifier when testing on an independent cohort. FIG. 3A is an ROC curve and FIG. 3B illustrates the NPV vs. TNR curve. The classifier is able to detect 50% of the non-responders with 100% accuracy.

FIGS. 4A-4D are plots illustrating in-cohort rheumatoid arthritis (RA) classifier validation using leave-one-out cross validation when training on Feature Set 1 (FIGS. 4A and 4B) and top nine signature genes (FIGS. 4C and 4D).

FIGS. 5A-5B are plots illustrating ROC curves of cross cohort classifier test results (in FIG. 5A) and negative predictive performance (in FIG. 5B) for the RA classifier.

FIG. 6 is an exemplary workflow for developing a classifier.

FIGS. 7A-7C provide identification of response discriminatory genes in cohort B. FIG. 7A provides Pearson correlation distribution of gene expression values with response outcomes in observed versus randomized gene expression data. The signal-to-noise ratio of actual and randomized Pearson correlations were derived by dividing the randomized valued by the observed value. FIG. 7B provides top 200 genes with highest signal-noise-ratio were mapped on the network resulting in observation of a significantly large connected component (LCC) shown in shaded region. FIG. 7C provides a heatmap representing the baseline gene expression values of LCC genes used for classifier training across patients. Red corresponds to higher relative expression values and yellow corresponds to lower relative expression values.

FIGS. 8A-8D provide cross-cohort performance of response prediction classifiers. FIG. 8A provides ROC curves of classifier validation in two independent cohorts. Classifier A is the classifier trained on cohort A and validated on cohort B and vice versa. FIG. 8B provides a depiction of accuracy in predicting non-responders (e.g., inadequate) responders to infliximab in an independent cohort. FIG. 8C provides classifier A prediction scores for cohort B patients. FIG. 8D provides Classifier B prediction scores for cohort A patients.

FIGS. 9A-9B provide distinct gene lists mapped onto the same network region of the Human Interactome indicated a common underlying biology of response. FIG. 9A illustrates largest connected component formed by the proteins encoded by the response signature genes from the two cohorts. Proteins encoded by cohort A genes are in orange and those encoded by cohort B genes are in blue. FIG. 9B illustrates distribution of LCC size from random expectation.

FIG. 10 is a map of the UC response module detected using the Human Interactome from cohort A. The response signature genes found in each cohort form a significant cluster (LCC) that is much bigger than expected by chance and reflects an underlying biology of response to infliximab in UC patients. Proteins are indicated by circles. Physical interactions are indicated by lines. Proteins encoded by the top 200 genes identified in each cluster that lack at least one physical interaction with a protein encoded by another top 200 gene are not shown.

FIG. 11 is an example network environment and computing devices for use in various embodiments.

FIG. 12 shows an example of a computing device 500 and a mobile computing device 550 that can be used to implement the techniques described in this disclosure.

### DETAILED DESCRIPTION

As noted, the response rate for patients undergoing anti-TNF therapy is inconsistent. Technologies that reliably identify responsive or non-responsive subjects would be beneficial, as they would avoid wasteful and even potentially damaging administration of therapy to subjects who will not respond, and furthermore would allow timely determination of more appropriate treatment for such subjects. The present disclosure provides such technologies, addressing needs of patients, their families, drug developers, and medical professionals each of whom suffers under the current system.

While significant effort has been invested in efforts to develop technologies that reliably predict responsiveness (e.g., by identifying responsive vs. non-responsive populations) or development of resistance for certain therapeutic agents, regimens, or modalities, success has been elusive, and almost exclusively limited to the oncology sector. Complex disorders, such as autoimmune and/or cardiovascular diseases have proven to be particularly challenging.

Cancer is typically associated with particular strong driver genes, which dramatically simplifies the analysis required to identify responder vs non-responder patient populations, and significantly improves success rates. By contrast, diseases associated with more complex genetic (and/or epigenetic) contributions, have thus far presented an insurmountable challenge for available technologies.

Indeed, a large number of published reports describe efforts to develop technologies for predicting responsiveness to anti-TNF therapy in inflammatory conditions (e.g., rheumatoid arthritis), most commonly relying on blood-based gene expression classifiers. See, e.g., Nakamura et al. “Identification of baseline gene expression signatures predicting therapeutic responses to three biologic agents in rheumatoid arthritis: a retrospective observational study”Arthritis Research & Therapy (2016) 18:159 DOI 10.1186/s13075-016-1052-8. However, a clinically utilizable classifier has not yet been identified. Notably, Toonen et al. performed an independent study that tested eight different gene expression signatures predicting response to anti-TNF, and reported that most signatures failed to demonstrate sufficient predictive value to be of utility. See M. Toonen et al., “Validation Study of Existing Gene Expression Signatures for Anti-TNF Treatment in Patients with Rheumatoid Arthritis,” PLOS ONE 7(3): e33199. Thomson et al. attempted to describe a blood-based classifier to identify non-responders to one anti-TNF therapy, infliximab, in rheumatoid arthritis. Thomson et al., “Blood-based identification of non-responders to anti-TNF therapy in rheumatoid arthritis,” BMC Med Genomics, 8:26, *1-12 (2015). Their proposed classifier comprised 18 signaling mechanisms indicative of higher TNF-mediated inflammatory signaling in responders at baseline, versus higher levels of specific metabolic activities in non-responders at baseline. The test, however, did not reach the level of predictive accuracy required for commercialization and so development was stopped.

Typically, conventional strategies for defining responder vs. non-responder classifiers for anti-TNF therapy rely on machine-learning approaches, using mean values across classes of response, and focusing on genes with the highest fold changes, often in a pathway-based context. The present disclosure identifies various sources of problems with these conventional approaches, and, moreover, provides technologies that solve or avoid the problems, thereby satisfying the long felt need within the community for accurate and/or useful predictive classifiers.

Among other things, the present disclosure appreciates that machine learning may be useful for finding correlation between datasets of patients, but fails to achieve sufficient predictive accuracy across cohorts. Furthermore, the present disclosure identifies that prioritizing or otherwise focusing on highest fold changes misses subtle but meaningful differences relevant to disease biology. Still further, the present disclosure offers an insight that mapping of genes with altered expression levels onto a human interactome (e.g., that represents experimentally supported physical interactions between cellular components and, in some embodiments, explicitly excludes any theoretical, calculated, or other interaction that has been proposed but not experimentally validated) can provide a useful and effective classifier for defining responders vs. non-responders to anti-TNF therapy. In some embodiments, genes included in such a classifier represent a connected module in the human interactome. Examples of methods of treatment and classifier development related to the present disclosure is found in WO 2019/178546, which is incorporated by reference herein in its entirety.

### Anti-TNF Therapy

TNF-mediated disorders are currently treated by inhibition of TNF, and in particular by administration of an anti-TNF agent (i.e., by anti-TNF therapy). Examples of anti-TNF agents approved for use in the United States include monoclonal antibodies such as adalimumab (Humira®), certolizumab pegol (Cimiza®), infliximab (Remicade), and decoy circulating receptor fusion proteins such as etanercept (Enbrel®). These agents are currently approved for use in treatment of indications, according to dosing regimens, as set forth below in Table 1:

TABLE 1

| Indication | Adalimumab¹ | Certolizumab Pegol¹ | Infliximab² | Etanercept¹ | Golimumab¹ | Golimumab² |
|---|---|---|---|---|---|---|
| Juvenile | 10 kg (22 lbs) to | N/A | N/A | 0.8 mg/kg weekly, | N/A | N/A |
| Idiopathic | <15 kg (33 lbs): | | | with a maximum | | |
| Arthritis | 10 mg every | | | of 50 mg per | | |
| | other week | | | week | | |
| | 15 kg (33 lbs) to | | | | | |
| | <30 kg (66 lbs): | | | | | |
| | 20 mg every | | | | | |
| | other week | | | | | |
| | ≥30 kg (66 lbs): | | | | | |
| | 40 mg every | | | | | |
| | other week | | | | | |
| Psoriatic | 40 mg every | 400 mg initially and at | 5 mg/kg at 0, 2 | 50 mg once weekly | 50 mg administered | N/A |
| Arthritis | other week | Weeks 2 and 4, | and 6 weeks, then | with or without | by subcutaneous | |
| | | followed by 200 mg | every 8 weeks | methotrexate | injection | |
| | | every other week; for | | | once a month | |
| | | maintenance dosing, | | | | |
| | | 400 mg every 4 weeks | | | | |
| Rheumatoid | 40 mg every | 400 mg initially and at | In conjunction with | 50 mg once weekly | 50 mg once a month | 2 mg/kg intravenous |
| Arthritis | other week | Weeks 2 and 4, | methotrexate, 3 | with or without | | infusion over 30 |
| | | followed by 200 mg | mg/kg at 0, 2 and | methotrexate | | minutes at weeks |
| | | every other week; for | 6 weeks, then | | | 0 and 4, then |
| | | maintenance dosing, | every 8 weeks | | | every 8 weeks |
| | | 400 mg every 4 weeks | | | | |
| Ankylosing | 40 mg every | 400 mg (given as 2 | 5 mg/kg at 0, 2 | 50 mg once weekly | 50 mg administered by | N/A |
| Spondylitis | other week | subcutaneous | and 6 weeks, then | | subcutaneous injection | |
| | | injections of 200 mg | every 6 weeks | | once a month | |
| | | each) initially and at | | | | |
| | | weeks 2 and 4, | | | | |
| | | followed by 200 mg | | | | |
| | | every other week or | | | | |
| | | 400 mg every 4 weeks | | | | |
| Adult | Initial dose (Day | 400 mg initially | 5 mg/kg at 0, 2 | N/A | N/A | N/A |
| Crohn's | 1): 160 mg | and at Weeks 2 | and 6 weeks, then | | | |
| Disease | Second dose two | and 4 | every 8 weeks. | | | |
| | weeks later (Day | Continue with 400 | | | | |
| | 15): 80 mg | mg every four | | | | |
| | Two weeks later | weeks | | | | |
| | (Day 29): Begin a | | | | | |
| | maintenance dose | | | | | |
| | of 40 mg every | | | | | |
| | other week | | | | | |
| Pediatric | 17 kg (37 lbs) to <40 | N/A | 5 mg/kg at 0, 2 | N/A | N/A | N/A |
| Crohn's | kg (88 lbs): | | and 6 weeks, then | | | |
| Disease | Initial dose (Day | | every 8 weeks. | | | |
| | 1): 80 mg | | | | | |
| | Second dose two | | | | | |
| | weeks later (Day | | | | | |
| | 15): 40 mg | | | | | |
| | Two weeks later | | | | | |
| | (Day 29): Begin a | | | | | |
| | maintenance dose | | | | | |
| | of 20 mg every | | | | | |
| | other week | | | | | |
| | ≥40 kg (88 lbs): | | | | | |
| | Initial dose (Day | | | | | |
| | 1): 160 mg | | | | | |
| | Second dose two | | | | | |
| | weeks later (Day | | | | | |
| | 15): 80 mg | | | | | |
| | Two weeks later | | | | | |
| | (Day 29): Begin a | | | | | |
| | maintenance dose | | | | | |
| | of 40 mg every | | | | | |
| | other week | | | | | |
| Ulcerative | Initial dose (Day | N/A | 5 mg/kg at 0, 2 | N/A | N/A | N/A |
| Colitis | 1): 160 mg | | and 6 weeks, then | | | |
| | Second dose two | | every 8 weeks. | | | |
| | weeks later (Day | | | | | |
| | 15): 80 mg | | | | | |
| | Two weeks later | | | | | |
| | (Day 29): Begin a | | | | | |
| | maintenance dose | | | | | |
| | of 40 mg every | | | | | |
| | other week | | | | | |
| Plaque | 80 mg initial dose; | N/A | N/A | 50 mg twice weekly | N/A | N/A |
| Psoriasis | 40 mg every other | | | for 3 months, | | |
| | week beginning one | | | followed by 50 mg | | |
| | week after initial | | | once weekly | | |
| | dose | | | | | |
| Hidradenitis | Initial dose (Day | N/A | N/A | N/A | N/A | N/A |
| Suppurativa | 1): 160 mg | | | | | |
| | Second dose two | | | | | |
| | weeks later (Day | | | | | |
| | 15): 80 mg | | | | | |
| | Third dose (Day | | | | | |
| | 29) and | | | | | |
| | subsequent doses: | | | | | |
| | 40 mg every week | | | | | |
| Uveitis | 80 mg initial dose; | N/A | N/A | N/A | N/A | N/A |
| | 40 mg every other | | | | | |
| | week beginning | | | | | |
| | one week after | | | | | |
| | initial dose | | | | | |

¹Administered by subcutaneous injection.
²Administered by intravenous infusion.

The present disclosure provides technologies relevant to anti-TNF therapy, including those therapeutic regimens as set forth in Table 1. In some embodiments, the anti-TNF therapy is or comprises administration of infliximab (Remicade®), adalimumab (Humira®), certolizumab pegol (Cimiza®), etanercept (Enbel®), or biosimilars thereof. In some embodiments, the anti-TNF therapy is or comprises administration of infliximab (Remicade®) or adalimumab (Humira®). In some embodiments, the anti-TNF therapy is or comprises administration of infliximab (Remicade®). In some embodiments, the anti-TNF therapy is or comprises administration of adalimumab (Humira®).

In some embodiments, the anti-TNF therapy is or comprises administration of a biosimilar anti-TNF agent. In some embodiments, the anti-TNF agent is selected from infliximab biosimilars such as CT-P13, BOW015, SB2, Inflectra, Renflexis, and Ixifi, adalimumab biosimilars such as ABP 501 (AMGEVITA™), Adfrar, and Hulio™ and etanercept biosimilars such as HD203, SB4 (Benepali®), GP2015, Erelzi®, and Intacept®.

In some embodiments, the present disclosure defines patient populations to whom anti-TNF therapy should (or should not) be administered. In some embodiments, technologies provided by the present disclosure generate information useful to doctors, pharmaceutical companies, payers, and/or regulatory agencies who wish to ensure that anti-TNF therapy is administered to responder populations and/or is not administered to non-responder populations.

### Diseases, Disorders or Conditions

In general, provided disclosures are useful in any context in which administration of anti-TNF therapy is contemplated or implemented. In some embodiments, provided technologies are useful in the diagnosis and/or treatment of subjects suffering from a disease, disorder, or condition associated with aberrant (e.g., elevated) TNF expression and/or activity. In some embodiments, provided technologies are useful in monitoring subjects who are receiving or have received anti-TNF therapy. In some embodiments, provided technologies identify whether a subject will or will not respond to a given anti-TNF therapy. In some embodiments, the provided technologies identify whether a subject will develop resistance to a given anti-TNF therapy.

Accordingly, the present disclosure provides technologies relevant to treatment of the various diseases, disorders, and conditions related to TNF, including those listed in Table 1. In some embodiments, a subject is suffering from a disease, disorder, or condition selected from rheumatoid arthritis, psoriatic arthritis, ankylosing spondylitis, Crohn's disease (adult or pediatric), ulcerative colitis, inflammatory bowel disease, chronic psoriasis, plaque psoriasis, hidradenitis suppurativa, asthma, uveitis, juvenile idiopathic arthritis, vitiligo, Graves' ophthalmopathy (also known as thyroid eye disease, or Graves' orbitopathy), and multiple sclerosis. In some embodiments, the disease, disorder, or condition is rheumatoid arthritis. In some embodiments, the disease, disorder, or condition is psoriatic arthritis. In some embodiments, the disease, disorder, or condition is ankylosing spondylitis. In some embodiments, the disease, disorder, or condition is Crohn's disease. In some embodiments, the disease, disorder, or condition is adult Crohn's disease. In some embodiments, the disease, disorder, or condition is pediatric Crohn's disease. In some embodiments, the disease, disorder, or condition is inflammatory bowel disease. In some embodiments, the disease, disorder, or condition is ulcerative colitis. In some embodiments, the disease, disorder, or condition is chronic psoriasis. In some embodiments, the disease, disorder, or condition is plaque psoriasis. In some embodiments, the disease, disorder, or condition is hidradenitis suppurativa. In some embodiments, the disease, disorder, or condition is asthma. In some embodiments, the disease, disorder, or condition is uveitis. In some embodiments, the disease, disorder, or condition is juvenile idiopathic arthritis. In some embodiments, the disease, disorder, or condition is vitiligo. In some embodiments, the disease, disorder, or condition is Graves' ophthalmopathy (also known as thyroid eye disease, or Graves' orbitopathy). In some embodiments, the disease, disorder, or condition is multiple sclerosis

### Provided Classifier(s)

The present disclosure provides classifiers that are or comprise gene expression response signatures that identify (i.e., predict) which patients will or will not respond to anti-TNF therapy. In some embodiments, a gene classifier comprises a gene expression response signature (e.g., a set of one or more genes) that distinguishes between responsive and non-responsive prior subjects (i.e., where “prior subjects” refers to subjects who have previously received an anti-TNF therapy, and have been classified as responders or non-responders).

As described herein, the present disclosure provides gene expression response signatures and methods for determining gene expression response signatures that are characteristic of anti-TNF responder or non-responder populations. In some embodiments, a particular gene expression response signature classifies responder or non-responder populations for a particular anti-TNF therapy (e.g., a particular anti-TNF agent and/or regimen). In some embodiments, a particular gene expression response signature classifies responder or non-responder populations suffering from a particular disease, disorder, or condition, for a particular anti-TNF therapy (e.g., a particular anti-TNF agent and/or regimen). In some embodiments, responder and/or non-responder populations for different anti-TNF therapies (e.g., different anti-TNF agents and/or regimens) may overlap or be co-extensive; in some such embodiments, the present disclosure may provide gene expression response signatures that serve as gene classifiers for responder and/or non-responder populations across anti-TNF therapies.

In some embodiments, as described herein, a gene expression response signature is identified by retrospective analysis of gene expression levels in biological samples from subjects who have received anti-TNF therapy (i.e., “prior subjects”) and have been determined to respond (i.e., are responders) or not to respond (i.e., are non-responders). In some embodiments, all such subjects have received the same anti-TNF therapy (optionally for the same or different periods of time); alternatively or additionally, in some embodiments, all such subjects have been diagnosed with the same disease, disorder or condition. In some embodiments, subjects whose biological samples are analyzed in the retrospective analysis had received different anti-TNF therapy (e.g., with a different anti-TNF agent and/or according to a different regimen); alternatively or additionally, in some embodiments, subjects whose biological samples are analyzed in the retrospective analysis have been diagnosed with different diseases, disorders, or conditions.

In some embodiments, a gene expression response signature as described herein is determined by comparison of gene expression levels in the responder vs. non-responder populations whose biological samples are analyzed in a retrospective analysis as described herein. In some embodiments, a gene expression response signature comprises genes whose individual expression levels show statistically significant differences between the responder and non-responder populations. In some embodiments, a gene expression response signature comprises genes whose linear combination of expression levels show statistically significant differences between the responder and non-responder populations. In some embodiments, a gene expression response signature comprises genes whose non-linear combination of expression levels show statistically significant differences between the responder and non-responder populations.

In some embodiments, a gene expression response signature is incorporated into a classifier for distinguishing between responder and non-responder subjects. In some embodiments, a classifier is developed by assessing each of: the one or more genes whose expression levels significantly correlate (e.g., in a linear and/or non-linear manner) to clinical responsiveness or non-responsiveness (i.e., a gene expression response signature); and optionally one or more of the presence of the one or more single nucleotide polymorphs (SNPs) and at least one clinical characteristic.

In some embodiments, the present disclosure embodies an insight that the source of a problem with certain prior efforts to identify or provide gene expression response signatures through comparison of gene expression levels in responder vs non-responder populations have emphasized and/or focused on (often solely on) genes that show the largest difference (e.g., greater than 2-fold change) in expression levels between the populations. The present disclosure appreciates that even genes those expression level differences are relatively small (e.g., less than 2-fold change in expression) provide useful information if the difference is significant, and are valuably included in a gene expression response signature in embodiments described herein.

Moreover, in some embodiments, the present disclosure embodies an insight that analysis of interaction patterns of genes whose expression levels show statistically significant differences (optionally including small differences) between responder and non-responder populations as described herein provides new and valuable information that materially improves the quality and predictive power of a gene expression response signature.

Further, as noted, the present disclosure provides technologies that allow practitioners to reliably and consistently predict response to anti-TNF therapy in a cohort of subjects (e.g., treatment naïve subjects, i.e., subjects who have not received anti-TNF therapy). In particular, for example, the rate of response for some anti-TNF therapies is less than 35% within a given cohort of subjects. The provided technologies allow for prediction of greater than 65% accuracy within a cohort of subjects a response rate (i.e., whether certain subjects will or will not respond to a given therapy). In some embodiments, the methods and systems described herein predict 65% or greater the subjects that are responders (i.e., will respond to anti-TNF therapy) within a given cohort. In some embodiments, the methods and systems described herein predict 70% or greater the subjects that are responders within a given cohort. In some embodiments, the methods and systems described herein predict 80% or greater the subjects that are responders within a given cohort. In some embodiments, the methods and systems described herein predict 90% or greater the subjects that are responders within a given cohort. In some embodiments, the methods and systems described herein predict 100% the subjects that are responders within a given cohort. In some embodiments, the methods and systems described herein predict 65% or greater the subjects that are non-responders (i.e., will not respond to anti-TNF therapy) within a given cohort. In some embodiments, the methods and systems described herein predict 70% or greater the subjects that are non-responders within a given cohort. In some embodiments, the methods and systems described herein predict 80% or greater the subjects that are non-responders within a given cohort. In some embodiments, the methods and systems described herein predict 90% or greater the subjects that are non-responders within a given cohort. In some embodiments, the methods and systems described herein predict 100% of the subjects that are non-responders within a given cohort.

In some embodiments, a gene expression response signature is developed by assessing one or more genes selected from Table A or Table B:

TABLE A

| | | | |
|---|---|---|---|
| ESR2 | MMP11 | SH2D3C | APBA1 |
| USPL1 | PRPS1 | CST1 | ING1 |
| SMARCA1 | LINC00672 | SDK1 | GSE1 |
| EFEMP2 | HDGFRP3 | LRRC23 | LOC100134040 |
| FTH1 | APOBR | LOC101929777 | VPREB3 |
| ASB16 | KIAA1107 | WDR24 | IFIH1 |
| PURA | BEX2 | HIST1H3F | INE1 |
| RUNX3 | CSRP2 | TAF10 | ARHGEF5 |
| BRF1 | RALGPS1 | ALDH1L1 | ECI1 |
| MAX | PSG5 | STRN3 | CKM |
| RBBP6 | TMEM135 | HAUS7 | ZNF711 |
| ARPC5L | SLC13A3 | PHKA2 | PCDHGA8 |
| MSH6 | CASP1 | SRI | RPS20 |
| SGCB | TYMP | SDHAP1 | NPIPA5 |
| SPAG9 | PPIAL4G | IGLL5 | ARHGEF12 |
| EDA | ST3GAL2 | RAC2 | PAGE2B |
| RABGEF1 | MRPS12 | CACNA1D | CSNK1G2 |
| FAM179B | TEK | CDC42SE2 | ARHGAP30 |
| HNRNPK | HOXB7 | NR2E3 | N4BP2L2 |
| HP1BP3 | RAB6B | TUBGCP6 | RBM34 |
| UBA2 | REEP3 | SUPV3L1 | PCDHGB3 |
| SFPQ | MFNG | INPP1 | GALNT8 |
| PKM | EFCAB13 | MR1 | ST7L |
| H3F3A | THAP11 | CASC4 | PSG6 |
| UBE2B | VIP | ZMYM5 | TBX3 |
| HRAS | TBCEL | TAPT1 | LRRC61 |
| HDAC4 | CYP2C19 | RASA4 | UPK3B |
| RBM26 | COL5A2 | C9orf163 | APOOL |
| ARF6 | GGT1 | PBDC1 | FGFR1 |
| MCM5 | SNORD68 | OR9A1P | RAB32 |
| ARNT | ADCY6 | NFKBIZ | SIRPA |
| HINFP | SLC13A3 | DGKG | CYP3A43 |
| SMC1A | COX15 | GNG10 | IGHG3 |
| MGST2 | YME1L1 | EVA1B | PISD |
| ATF7IP | UGT1A7 | FGFR3 | LINC01279 |
| CHFR | ZNF800 | 15-Sep | MIR1302-9 |
| MED6 | LRP3 | CCDC105 | SIPA1L1 |
| ATP6V0C | MMP11 | RSBN1 | CDT1 |
| SUMO2 | GEMIN2 | ITPR1 | WSB1 |
| RECQL | ANKHD1 | PTPRN2 | KLHL26 |
| ARCN1 | CYP4X1 | TMEM248 | PLGLB1 |
| CEACAM1 | MACROD1 | NAA40 | SKAP2 |
| KCNE3 | HMGA1 | SEC31A | MMP15 |
| PMEPA1 | STX5 | COL1A1 | TRIL |
| C9orf16 | PAK2 | PRR4 | SMARCD2 |
| ATP6AP1 | BMP5 | NOTCH3 | IL9 |
| LINC00910 | STXBP3 | DCLREIC | |
| SERPINB8 | ST8SIA1 | VAPB | |
| MS4A1 | DHRS3 | SLC39A8 | |

TABLE B

| | | | |
|---|---|---|---|
| TFIP11 | SBF2 | TBKBP1 | KIR2DL3 |
| MDM2 | EFNB3 | CR2 | ATP2C1 |
| PML | CNTD1 | LRRC23 | CSF2 |
| SNRPN | ARG2 | TNRC6B | DRD4 |
| NFAT5 | TRHDE-AS1 | NOP10 | ZNF696 |
| PNN | SOX17 | DDR2 | TXNL4A |
| TRAPPC4 | SUN1 | IDUA | LOC102723661 |
| RRP15 | NUDT4P1 | CTSO | MAP4K1 |
| PRKAB1 | LOC145783 | ARHGEF40 | HMGB3P1 |
| ERICH1 | RAPH1 | KCNQ1-AS1 | LOC100996792 |
| LCA5 | TMEM119 | NR2F6 | VSTM2A |
| CIRBP | GTF2IP4 | DBNDD1 | GRM7 |
| HHEX | ATP2A3 | PKIA | OXA1L |
| YWHAE | MLLT3 | HIST1H4K | SLC33A1 |
| UBA5 | LOC101928955 | HOXC6 | KLF8 |
| BRD7 | HIST1H4H | ALDHIL1 | FAM195B |
| ATRX | TNFRSF10C | WHAMMP3 | PCDHGA6 |
| EEA1 | LCP2 | MROH8 | CCDC97 |
| NUCKS1 | PCDHGA1 | COL9A3 | THAP6 |
| KLF3 | VASH2 | MIR9-2 | SDS |
| FAM207A | RAB11FIP4 | DET1 | TMCO2 |
| ANP32B | RAPGEF3 | IMPACT | KLK15 |
| SUMO2 | VN1R3 | PGM2 | KAT7 |
| CLTC | TUSC2 | C1QTNF3 | SRD5A3-AS1 |
| CGN | LRRC27 | NSUN4 | MLX |
| FAM192A | DCAF16 | ARMCX2 | APOL1 |
| CFAP206 | AVL9 | KCNE3 | B4GALT4 |
| CCDC88A | C3orf52 | PCCA | KIAA0430 |
| CAPN1 | GPALPP1 | MXRA7 | CNPY2 |
| VPS72 | AQP6 | DNAJB11 | CYP3A4 |
| TNK2 | ZNF551 | TSPEAR-AS2 | FBXO31 |
| RBCK1 | PON3 | MIR4680 | OR2B2 |
| TPR | UGT1A3 | ZNF638 | |
| PKM | EPPIN-WFDC6 | PPP1R9A | |
| MDC1 | CDKL2 | AGFG2 | |
| THTPA | EIF4EBP3 | SNORD68 | |
| UBE2D1 | APBA3 | MIDN | |
| TMEM87A | PBK | SNORD107 | |
| ADAR | COL25A1 | UXS1 | |
| ANKRD26 | GTF2H1 | RASA4 | |
| RAD51AP1 | MAGI2-AS3 | FAM199X | |
| BACH2 | FBLN1 | GAGE12G | |
| ABI3BP | FBXO31 | GRAP | |
| HIST1H4C | CSTF2 | RGS7 | |
| ZFHX4 | PDGFD | SDAD1 | |
| CCDC144A | SLC2A13 | GTF3C6 | |
| SNORD73A | SNORD58A | KIR3DL1 | |
| MIR7113 | MZB1 | SUN5 | |
| SEMA4F | MMP11 | HMX1 | |
| EFNA5 | CTNNA3 | PAPLN | |

In some embodiments, a gene expression response signature is developed by assessing one or more genes selected from Table C and Table D:

TABLE C

ESR2
USPL1
SMARCA1
EFEMP2
FTH1
ASB16
PURA
RUNX3
BRF1
MAX
RBBP6
ARPC5L
MSH6
SGCB
SPAG9
EDA
RABGEF1
FAM179B
HNRNPK
HP1BP3
UBA2
SFPQ
PKM
H3F3A
UBE2B
HRAS
HDAC4
RBM26
ARF6
MCM5
ARNT
HINFP
SMC1A
MGST2
ATF7IP
CHFR
MED6
ATP6V0C
SUMO2
RECQL
ARCN1

TABLE D

TFIP11
MDM2
PML
SNRPN
NFAT5
PNN
TRAPPC4
RRP15
PRKAB1
ERICH1
LCA5
CIRBP
HHEX
YWHAE
UBA5
BRD7
ATRX
EEA1
NUCKS1
KLF3
FAM207A
ANP32B
SUMO2
CLTC
CGN
FAM192A
CFAP206
CCDC88A
CAPN1
VPS72
TNK2
RBCK1
TPR
PKM
MDC1
THTPA
UBE2D1
TMEM87A
ADAR

In some embodiments, a gene expression response signature is developed by assessing one or more genes selected from Table E:

TABLE E

| | |
|---|---|
| ESR2 | ARCN1 |
| USPL1 | TFIP11 |
| SMARCA1 | MDM2 |
| EFEMP2 | PML |
| FTH1 | SNRPN |
| ASB16 | NFAT5 |
| PURA | PNN |
| RUNX3 | TRAPPC4 |
| BRF1 | RRP15 |
| MAX | PRKAB1 |
| RBBP6 | ERICH1 |
| ARPC5L | LCA5 |
| MSH6 | CIRBP |
| SGCB | HHEX |
| SPAG9 | YWHAE |
| EDA | UBA5 |
| RABGEF1 | BRD7 |
| FAM179B | ATRX |
| HNRNPK | EEA1 |
| HP1BP3 | NUCKS1 |
| UBA2 | KLF3 |
| SFPQ | FAM207A |
| PKM | ANP32B |
| H3F3A | SUMO2 |
| UBE2B | CLTC |
| HRAS | CGN |
| HDAC4 | FAM192A |
| RBM26 | CFAP206 |
| ARF6 | CCDC88A |
| MCM5 | CAPN1 |
| ARNT | VPS72 |
| HINFP | TNK2 |
| SMC1A | RBCK1 |
| MGST2 | TPR |
| ATF7IP | PKM |
| CHFR | MDC1 |
| MED6 | THTPA |
| ATP6V0C | UBE2D1 |
| SUMO2 | TMEM87A |
| RECQL | ADAR |

In some embodiments, a gene expression response signature is developed by assessing SUMO2 and/or PKM.

### Defining Classifier(s)

A provided gene expression response signature is a gene or set of genes that can be used to determine whether a subject will or will not respond to a particular therapy (e.g., anti-TNF therapy). A gene expression response signature itself can be a classifier, or can otherwise be part of a classifier that distinguishes between responsive and non-responsive subjects. In some embodiments, a gene expression response signature can be identified using mRNA and/or protein expression datasets, for example as may be or have been prepared from validated biological data (e.g., biological data derived from publicly available databases such as Gene Expression Omnibus (“GEO”)). In some embodiments, a gene expression response signature may be derived by comparing gene expression levels of known responsive and known non-responsive prior subjects to a specific therapy (e.g., anti-TNF therapy). In some embodiments, certain genes (i.e., signature genes) are selected from this cohort of gene expression data to be used in developing the gene expression response signature.

In some embodiments, signature genes are identified by methods analogous to those reported by Santolini, “A personalized, multiomics approach identifies genes involved in cardiac hypertrophy and heart failure,” Systems Biology and Applications, (2018)4:12; doi:10.1038/s41540-018-0046-3, which is incorporated herein by reference. In some embodiments, signature genes are identified by comparing gene expression levels of known responsive and non-responsive prior subjects and identifying significant changes between the two groups, wherein the significant changes can be large differences in expression (e.g., greater than 2-fold change), small differences in expression (e.g., less than 2-fold change), or both. In some embodiments, genes are ranked by significance of difference in expression. In some embodiments, significance is measured by Pearson correlation between gene expression and response outcome. In some embodiments, signature genes are selected from the ranking by significance of difference in expression. In some embodiments, the number of signature genes selected is less than the total number of genes analyzed. In some embodiments, 200 signature genes or less are selected. In some embodiments 100 genes or less are selected.

In some embodiments, signature genes are selected in conjunction with their location on a human interactome (HI), a map of protein-protein interactions. Use of the HI in this way encompasses a recognition that mRNA activity is dynamic and determines the actual over and under expression of proteins critical to understanding certain diseases. In some embodiments, genes associated with response to certain therapies (i.e., anti-TNF therapy) may cluster (i.e., form a cluster of genes) in discrete modules on the HI map. The existence of such clusters is associated with the existence of fundamental underlying disease biology. In some embodiments, a gene expression response signature is derived from signature genes selected from the cluster of genes on the HI map. Accordingly, in some embodiments, a gene expression response signature is derived from a cluster of genes associated with response to anti-TNF therapy on a human interactome map.

In some embodiments, genes associated with response to certain therapies exhibit certain topological properties when mapped onto a human interactome map. For example, in some embodiments, a plurality of genes associated with response to anti-TNF therapy and characterized by their position (i.e., topological properties, e.g., their proximity to one another) on a human interactome map.

In some embodiments, genes associated with response to certain therapies (i.e., anti-TNF therapy) may exist within close proximity to one another on the HI map. Said proximal genes, do not necessarily need to share fundamental underlying disease biology. That is, in some embodiments, proximal genes do not share significant protein interaction. Accordingly, in some embodiments, the gene expression response signature is derived from genes that are proximal on a human interactome map. In some embodiments, the gene expression response signature is derived from certain other topological features on a human interactome map.

In some embodiments, genes associated with response to certain therapies (i.e., anti-TNF therapy) may be determined by Diffusion State Distance (DSD) (see Cao, et al., PLOS One, 8(10): e76339 (Oct. 23, 2013)) when used in combination with the HI map.

In some embodiments, signature genes are selected by (1) ranking genes based on the significance of difference of expression of genes as compared to known responders and known non-responders; (2) selecting genes from the ranked genes and mapping the selected genes onto a human interactome map; and (3) selecting signature genes from the genes mapped onto the human interactome map.

In some embodiments, signature genes (e.g., selected from the Santolini method, or using various network topological properties including, but not limited to, clustering, proximity and diffusion-based methods) are provided to a probabilistic neural network to thereby provide (i.e., “train”) the gene expression response signature. In some embodiments, the probabilistic neural network implements the algorithm proposed by D. F. Specht in “Probabilistic Neural Networks,” Neural Networks, 3(1):109-118 (1990), which is incorporated herein by reference. In some embodiments, the probabilistic neural network is written in the R-statistical language, and knowing a set of observations described by a vector of quantitative variables, classifies observations into a given number of groups (e.g., responders and non-responders). The algorithm is trained with the data set of signature genes taken from known responders and non-responders and guesses new observations that are provided. In some embodiments, the probabilistic neural network is one derived from the Comprehensive R Network.

Alternatively or additionally, in some embodiments, a gene expression response signature can be trained in the probabilistic neural network using a cohort of known responders and non-responders using leave-one-out cross and/or k-fold cross validation. In some embodiments, such a process leaves one sample out (i.e., leave-one-out) of the analysis and trains the classifier only based on the remaining samples. In some embodiments, the updated classifier is then used to predict a probability of response for the sample that's left out. In some embodiments, such a process can be repeated iteratively, for example, until all samples have been left out once. In some embodiments, such a process randomly partitions a cohort of known responders and non-responders into k equal sizes groups. Of the k groups, a single group is retained as validation data for testing the model, and the remaining groups are used as training data. Such a process can be repeated k times, with each of the k groups being used exactly once as the validation data. In some embodiments, the outcome is a probability score for each sample in the training set. Such probability scores can correlate with actual response outcome. A Recursive Operating Curves (ROC) can be used to estimate the performance of the classifier. In some embodiments, an Area Under Curve (AUC) of about 0.6 or higher reflects a suitably validated classifier. In some embodiments, a Negative Predictive Value (NPV) of 0.9 reflects a suitable validated classifier. In some embodiments, a classifier can be tested in a completely independent (i.e., blinded) cohort to, for example, confirm the suitability (i.e., using leave-one-out and/or k-fold cross validation). Accordingly, in some embodiments, provided methods further comprise one or more steps of validating a gene expression response signature, for example, by assigning probability of response to a group of known responders and non-responders; and checking the gene expression response signature against a blinded group of responders and non-responders. The output of these processes is a trained gene expression response signature useful for establishing whether a subject will or will not respond to a particular therapy (e.g., anti-TNF therapy).

In some embodiments, a gene expression response signature is validated using a cohort of subjects having previously been treated with anti-TNF therapy, but is independent from the cohort of subjects used to prepare the classifier. In some embodiments, a gene expression response signature is considered “validated” when 90% or greater of non-responding subjects are predicted with 50% or greater accuracy within the validating cohort.

In some embodiments, the gene expression response signature predicts responsiveness of subjects with at least 50% accuracy across a population of subjects. In some embodiments, the gene expression response signature predicts responsiveness of subjects with at least 60% accuracy predicting responsiveness across a population of subjects. In some embodiments, the gene expression response signature predicts responsiveness of subjects with at least 80% accuracy across a population of subjects. In some embodiments, the gene expression response signature predicts responsiveness of subjects with at least 90% accuracy across a population of subjects. In some embodiments, the gene expression response signature predicts responsiveness of subjects with at least 95% accuracy across a population of subjects. In some embodiments, the gene expression response signature predicts responsiveness of subjects with at least 97% accuracy across a population of subjects. In some embodiments, the gene expression response signature predicts responsiveness of subjects with at least 98% accuracy across a population of subjects. In some embodiments, the gene expression response signature predicts responsiveness of subjects with at least 99% accuracy across a population of subjects.

Accordingly, in some embodiments, the gene expression response signature is established to distinguish between responsive and non-responsive prior subjects who have received a type of therapy, e.g., anti-TNF therapy. This gene expression response signature, derived from these prior responders and non-responders, is used to classify subjects (outside of the previously-identify cohorts) as responders or non-responders, i.e., can predict whether a subject will or will not respond to a given therapy. In some embodiments, the response and non-responsive prior subjects suffered from the same disease, disorder, or condition.

In some embodiments, a classifier is validated by analyzing gene expression levels in biological samples from a first cohort of subjects who have previously received the anti-TNF therapy (“prior subjects”) and have been determined to respond (“responders”) or not to respond (“non-responders”) to the anti-TNF therapy to identify genes that show statistically significant differences in expression level between the responders and the non-responders (“signature genes”). In some embodiments, signature genes are mapped onto a biological network (e.g., a human interactome). In some embodiments, a subset of signature genes are selected on the basis of their connectivity in the biological network to provide a candidate gene list. In some embodiments, a method of validating a classifier comprising training a classifier (e.g., an non-validated classifier) on expression levels of the genes of the candidate gene list from the first cohort of subjects (e.g., prior subjects, that is, subjects who have previously been classified as responsive or non-responsive to anti-TNF therapy) to identify a subset of the prior subjects having a pattern of expression of the candidate gene list indicative that the subset of prior subjects are unlikely to respond to the anti-TNF therapy, to thereby obtain a trained classifier.

In some embodiments, a trained classifier is validated via analysis of a second cohort comprising an independent and blinded group of responders and non-responders, and selecting a cutoff score such that the validated classifier distinguishes about 50% of prior subjects that are non-responsive (i.e., have a TNR of about 0.5) to the anti-TNF therapy. In some embodiments, a validated classifier distinguishes about 65% of prior subjects that are non-responsive (i.e., have a TNR of about 0.65) to the anti-TNF therapy. In some embodiments, a validated classifier distinguishes about 70% of prior subjects that are non-responsive (i.e., have a TNR of about 0.7) to the anti-TNF therapy. In some embodiments, a validated classifier distinguishes about 80% of prior subjects that are non-responsive (i.e., have a TNR of about 0.8) to the anti-TNF therapy. In some embodiments, a validated classifier distinguishes about 90% of prior subjects that are non-responsive (i.e., have a TNR of about 0.9) to the anti-TNF therapy. In some embodiments, a validated classifier distinguishes about 95% of prior subjects that are non-responsive (i.e., have a TNR of about 0.95) to the anti-TNF therapy. In some embodiments, a validated classifier distinguishes about 100% of prior subjects that are non-responsive (i.e., have a TNR of about 1.0) to the anti-TNF therapy.

In some embodiments, a validated classifier distinguishes at least 50% of prior subjects that are non-responsive to the anti-TNF therapy with at least 60% NPV (i.e., has an NPV of about 0.6). In some embodiments, a validated classifier distinguishes at least 50% of prior subjects that are non-responsive to the anti-TNF therapy with at least 70% NPV (i.e., has an NPV of about 0.7). In some embodiments, a validated classifier distinguishes at least 50% of prior subjects that are non-responsive to the anti-TNF therapy with at least 80% NPV (i.e., has an NPV of about 0.8). In some embodiments, a validated classifier distinguishes at least 50% of prior subjects that are non-responsive to the anti-TNF therapy with at least 90% NPV (i.e., has an NPV of about 0.9). In some embodiments, a validated classifier distinguishes at least 50% of prior subjects that are non-responsive to the anti-TNF therapy with at least 95% NPV (i.e., has an NPV of about 0.95). In some embodiments, a validated classifier distinguishes at least 50% of prior subjects that are non-responsive to the anti-TNF therapy with at least 100% NPV (i.e., has an NPV of about 1.0).

### Detecting Classifier(s)

Detecting gene classifiers in subjects, once the gene classifier is identified, is a routine matter for those of skill in the art. In other words, by first defining the gene classifier, a variety of methods can be used to determine whether a subject or group of subjects express the established gene classifier. For example, in some embodiments, a practitioner can obtain a blood or tissue sample from the subject prior to administering of therapy, and extract and analyze mRNA profiles from said blood or tissue sample. The analysis of gene expression profiles can be performed by any method known to those of skill in the art, including, but not limited hybridization-based RNA detection assays (such as assays based on microarray, bead array, and NANOSTRING (direct detection of color-coded hybridized probes) technologies), RNA sequencing assays, amplification-based RNA detection assays (such as real-time quantitative reverse transcription polymerase chain reaction (qRT-PCR) or reverse transcription loop mediated isothermal amplification (RT-LAMP)), mass spectrometry-based protein detection assays (such as targeted mass spectrometry (MRM or SRM) or immunoaffinity liquid chromatography—tandem mass spectrometry (IA LC-MS/MC)) and immunoassay-based protein detection assays (such as enzyme-linked immunosorbent assays (ELISA), immunohistochemistry, or flow cytometry). Accordingly, in some embodiments, the present disclosure provides methods of determining whether a subject is classified as a responder or non-responder, comprising measuring gene expression by at least one of a microarray, RNA sequencing, real-time quantitative reverse transcription PCR (qRT-PCR), bead array, and ELISA. In some embodiments, the present disclosure provides methods of determining whether a subject is classified as a responder or non-responder comprising measuring gene expression of a subject by RNA sequencing (i.e., RNAseq).

In some embodiments, the provided technologies provide methods comprising determining, prior to administering anti-TNF therapy, that a subject displays a gene expression response signature associated with response to anti-TNF therapy; and administering the anti-TNF therapy to the subject determined to display the gene expression response signature. In some embodiments, the provided technologies provide methods comprising determining, prior to administering anti-TNF therapy, that a subject does not display the gene expression response signature; and administering a therapy alternative to anti-TNF therapy to the subject determine not to display the gene expression signature.

In some embodiments, the therapy alternative to anti-TNF therapy is selected from rituximab (Rituxan®), sarilumab (Kevzara®), tofacitinib citrate (Xeljanz®), leflunomide (Arava®), vedolizumab (Entyvio®), tocilizumab (Actemra®), anakinra (Kineret®), and abatacept (Orencia®).

In some embodiments, gene expression is measured by subtracting background data, correcting for batch effects, and dividing by mean expression of housekeeping genes. See Eisenberg & Levanon, “Human housekeeping genes, revisited,” Trends in Genetics, 29(10):569-574 (Oct. 2013). In the context of microarray data analysis, background subtraction refers to subtracting the average fluorescent signal arising from probe features on a chip not complimentary to any mRNA sequence, i.e. signals that arise from non-specific binding, from the fluorescence signal intensity of each probe feature. The background subtraction can be performed with different software packages, such as Affymetrix® Gene Expression Console. Housekeeping genes are involved in basic cell maintenance and, therefore, are expected to maintain constant expression levels in all cells and conditions. The expression level of genes of interest, i.e., those in the response signature, can be normalized by dividing the expression level by the average expression level across a group of selected housekeeping genes. This housekeeping gene normalization procedure calibrates the gene expression level for experimental variability. Further, normalization methods such as robust multi-array average (“RMA”) correct for variability across different batches of microarrays, are available in R packages recommended by either Illumina® and/or Affymetrix® platforms. The normalized data is log transformed, and probes with low detection rates across samples are removed. Furthermore, probes with no available genes symbol or Entrez ID are removed from the analysis.

In some embodiments, the present disclosure provides a kit comprising means for detecting a gene expression response signature established to distinguish between responsive and non-responsive prior subjects who have received anti-TNF therapy. In some embodiments, the kit facilitates comparison levels of gene expression of a subject to the gene expression response signature (i.e., the gene classifier) established to distinguish between responsive and non-responsive prior subjects who have received anti-TNF therapy. In some embodiments, a kit comprises a set of reagents for detecting an expression level of one or more genes in a gene expression response signature described herein.

In some embodiments, the present disclosure provides a kit comprising means for detecting a gene expression response signature established to distinguish between responsive and non-responsive prior subjects suffering from a disease, disorder, or condition and who have received anti-TNF therapy, wherein the gene expression response signature comprises an expression level of PKM and SUMO2.

In some embodiments, the present disclosure provides a kit for evaluating a likelihood that a patient having an autoimmune disorder will not respond to an anti-TNF therapy, the kit comprising a set of reagents for detecting an expression level of one or more genes selected from the group consisting of: PKM, ADAR, ANP32B, ATRX, BRD7, CAPN1, CCDC88A, CFAP206, CGN, CIRBP, CLTC, EEA1, ERICH1, FAM192A, FAM207A, HHEX, KLF3, LCA5, MDC1, MDM2, NFAT5, ARCN1, ARF6, ARNT, ARPC5L, ASB16, ATF7IP, ATP6VOC, BRF1, CHFR, EDA, EFEMP2, ESR2, FAM179B, FTH1, H3F3A, HDAC4, HINFP, HNRNPK, SUMO2, NUCKS1, PML, PNN, PRKAB1, RBCK1, RRP15, SNRPN, TFIP11, THTPA, TMEM87A, TNK2, TPR, TRAPPC4, UBA5, UBE2D1, VPS72, YWHAE, MCM5, MED6, MGST2, MSH6, PURA, RABGEF1, RBBP6, RBM26, RECQL, RUNX3, SFPQ, SGCB, SMARCA1, SMC1A, SPAG9, UBA2, UBE2B, USPL1, HP1BP3, HRAS, or MAX.

As described herein, a kit comprises a set of reagents for detecting and/or measuring expression level of one or more genes described herein. In some embodiments, a kit comprises components for hybridization-based RNA detection assays (such as assays based on microarray, bead array, and NANOSTRING (direct detection of color-coded hybridized probes) technologies), RNA sequencing assays, amplification-based RNA detection assays (such as real-time quantitative reverse transcription polymerase chain reaction (qRT-PCR) or reverse transcription loop mediated isothermal amplification (RT-LAMP)), mass spectrometry-based protein detection assays (such as targeted mass spectrometry (MRM or SRM) or immunoaffinity liquid chromatography—tandem mass spectrometry (IA LC-MS/MC)) and immunoassay-based protein detection assays (such as enzyme-linked immunosorbent assays (ELISA), immunohistochemistry, or flow cytometry).

In some embodiments, the gene expression response signature comprises an expression level of (1) PKM and SUMO2; and (2) one or more genes selected from: ADAR, ANP32B, ATRX, BRD7, CAPN1, CCDC88A, CFAP206, CGN, CIRBP, CLTC, EEA1, ERICH1, FAM192A, FAM207A, HHEX, KLF3, LCA5, MDC1, MDM2, NFAT5, NUCKS1, PML, PNN, PRKAB1, RBCK1, RRP15, SNRPN, TFIP11, THTPA, TMEM87A, TNK2, TPR, TRAPPC4, UBA5, UBE2D1, VPS72, or YWHAE.

In some embodiments the gene expression response signature comprises an expression level of (1) PKM and SUMO2; and (2) one or more genes selected from: ARCN1, ARF6, ARNT, ARPC5L, ASB16, ATF7IP, ATP6VOC, BRF1, CHFR, EDA, EFEMP2, ESR2, FAM179B, FTH1, H3F3A, HDAC4, HINFP, HNRNPK, HP1BP3, HRAS, MAX, MCM5, MED6, MGST2, MSH6, PURA, RABGEF1, RBBP6, RBM26, RECQL, RUNX3, SFPQ, SGCB, SMARCA1, SMC1A, SPAG9, UBA2, UBE2B, or USPL1.

In some embodiments, the gene expression response signature comprises an expression level of (1) PKM and SUMO2; and (2) one or more genes selected from: ADAR, ANP32B, ATRX, BRD7, CAPN1, CCDC88A, CFAP206, CGN, CIRBP, CLTC, EEA1, ERICH1, FAM192A, FAM207A, HHEX, KLF3, LCA5, MDC1, MDM2, NFAT5, ARCN1, ARF6, ARNT, ARPC5L, ASB16, ATF7IP, ATP6VOC, BRF1, CHFR, EDA, EFEMP2, ESR2, FAM179B, FTH1, H3F3A, HDAC4, HINFP, HNRNPK, NUCKS1, PML, PNN, PRKAB1, RBCK1, RRP15, SNRPN, TFIP11, THTPA, TMEM87A, TNK2, TPR, TRAPPC4, UBA5, UBE2D1, VPS72, YWHAE, MCM5, MED6, MGST2, MSH6, PURA, RABGEF1, RBBP6, RBM26, RECQL, RUNX3, SFPQ, SGCB, SMARCA1, SMC1A, SPAG9, UBA2, UBE2B, USPL1, HP1BP3, HRAS, or MAX.

### Using Classifiers

Patient Stratification

Among other things, the present disclosure provides technologies for predicting responsiveness to anti-TNF therapies. In some embodiments, provided technologies exhibit consistency and/or accuracy across cohorts superior to previous methodologies.

Thus, the present disclosure provides technologies for patient stratification, defining and/or distinguishing between responder and non-responder populations. For example, in some embodiments, the present disclosure provides methods for treating subjects with anti-TNF therapy, which methods, in some embodiments, comprise a step of: administering the anti-TNF therapy to subjects who have been determined not to display a gene expression response signature established to distinguish between responsive and non-responsive prior subjects who have received the anti-TNF therapy. In some such embodiments, the gene expression response signature includes a plurality of genes established to distinguish between responsive and non-responsive prior subjects for a given anti-TNF therapy. In some embodiments, the plurality of genes are determined to cluster with one another in a human interactome map. In some embodiments, the plurality of genes are proximal in a human interactome map. In some embodiments, the plurality of genes comprise genes that are shown to be statistically significantly different between responsive and non-responsive prior subjects.

Methods of Treatment and Therapy Monitoring

Further, the present disclosure provides technologies for monitoring therapy for a given subject or cohort of subjects. As a subject's gene expression level can change over time, it may, in some instances, be necessary or desirable to evaluate a subject at one or more points in time, for example, at specified and or periodic intervals.

In some embodiments, the present disclosure provides a method of treating a subject suffering from a disease, disorder, or condition (e.g., inflammatory bowel disease, ulcerative colitis or Crohn's disease) with an anti-TNF therapy, the method comprising a step of: administering the anti-TNF therapy to subjects who have been determined not to display a gene expression response signature established to distinguish between responsive and non-responsive prior subjects who have received the anti-TNF therapy, wherein the gene expression response signature comprises an expression level of PKM and SUMO2.

In some embodiments, the present disclosure provides A method of treating a subject suffering from a disease, disorder, or condition with an anti-TNF therapy, the method comprising a step of: administering the anti-TNF therapy to subjects who have been determined to be responsive via a classifier determined to distinguish between responsive and non-responsive subjects who have received the anti-TNF therapy (“prior subject”), and the classifier measures expression of one or more genes (e.g., two or more, three or more, four or more, five or more, six or more, or substantially all) selected from: PKM, ADAR, ANP32B, ATRX, BRD7, CAPN1, CCDC88A, CFAP206, CGN, CIRBP, CLTC, EEA1, ERICH1, FAM192A, FAM207A, HHEX, KLF3, LCA5, MDC1, MDM2, NFAT5, ARCN1, ARF6, ARNT, ARPC5L, ASB16, ATF7IP, ATP6VOC, BRF1, CHFR, EDA, EFEMP2, ESR2, FAM179B, FTH1, H3F3A, HDAC4, HINFP, HNRNPK, SUMO2, NUCKS1, PML, PNN, PRKAB1, RBCK1, RRP15, SNRPN, TFIP11, THTPA, TMEM87A, TNK2, TPR, TRAPPC4, UBA5, UBE2D1, VPS72, YWHAE, MCM5, MED6, MGST2, MSH6, PURA, RABGEF1, RBBP6, RBM26, RECQL, RUNX3, SFPQ, SGCB, SMARCA1, SMC1A, SPAG9, UBA2, UBE2B, USPL1, HP1BP3, HRAS, or MAX.

In some embodiments, the classifier measures expression of one or more genes (e.g., two or more, three or more, four or more, five or more, six or more, or substantially all) selected from: SUMO2, ADAR, ANP32B, ATRX, BRD7, CAPN1, CCDC88A, CFAP206, CGN, CIRBP, CLTC, EEA1, ERICH1, FAM192A, FAM207A, HHEX, KLF3, LCA5, MDC1, MDM2, NFAT5, PKM, NUCKS1, PML, PNN, PRKAB1, RBCK1, RRP15, SNRPN, TFIP11, THTPA, TMEM87A, TNK2, TPR, TRAPPC4, UBA5, UBE2D1, VPS72, or YWHAE.

In some embodiments, the classifier measures expression of one or more genes (e.g., two or more, three or more, four or more, five or more, six or more, or substantially all) selected from: SUMO2, ARCN1, ARF6, ARNT, ARPC5L, ASB16, ATF7IP, ATP6VOC, BRF1, CHFR, EDA, EFEMP2, ESR2, FAM179B, FTH1, H3F3A, HDAC4, HINFP, HNRNPK, HP1BP3, HRAS, MAX, PKM, MCM5, MED6, MGST2, MSH6, PURA, RABGEF1, RBBP6, RBM26, RECQL, RUNX3, SFPQ, SGCB, SMARCA1, SMC1A, SPAG9, UBA2, UBE2B, or USPL1.

In some embodiments, the classifier measures expression of SUMO2 and PKM.

In some embodiments, the classifier measures expression levels of two or more genes selected from: PKM, ADAR, ANP32B, ATRX, BRD7, CAPN1, CCDC88A, CFAP206, CGN, CIRBP, CLTC, EEA1, ERICH1, FAM192A, FAM207A, HHEX, KLF3, LCA5, MDC1, MDM2, NFAT5, ARCN1, ARF6, ARNT, ARPC5L, ASB16, ATF7IP, ATP6VOC, BRF1, CHFR, EDA, EFEMP2, ESR2, FAM179B, FTH1, H3F3A, HDAC4, HINFP, HNRNPK, SUMO2, NUCKS1, PML, PNN, PRKAB1, RBCK1, RRP15, SNRPN, TFIP11, THTPA, TMEM87A, TNK2, TPR, TRAPPC4, UBA5, UBE2D1, VPS72, YWHAE, MCM5, MED6, MGST2, MSH6, PURA, RABGEF1, RBBP6, RBM26, RECQL, RUNX3, SFPQ, SGCB, SMARCA1, SMC1A, SPAG9, UBA2, UBE2B, USPL1, HP1BP3, HRAS, or MAX.

In some embodiments, a gene expression response signature comprises an expression level of (1) PKM and SUMO2, and (2) one or more genes selected from: ADAR, ANP32B, ATRX, BRD7, CAPN1, CCDC88A, CFAP206, CGN, CIRBP, CLTC, EEA1, ERICH1, FAM192A, FAM207A, HHEX, KLF3, LCA5, MDC1, MDM2, NFAT5, NUCKS1, PML, PNN, PRKAB1, RBCK1, RRP15, SNRPN, TFIP11, THTPA, TMEM87A, TNK2, TPR, TRAPPC4, UBA5, UBE2D1, VPS72, or YWHAE.

In some embodiments, a gene expression response signature comprises an expression level of (1) PKM and SUMO2, and (2) one or more genes selected from: ARCN1, ARF6, ARNT, ARPC5L, ASB16, ATF7IP, ATP6VOC, BRF1, CHFR, EDA, EFEMP2, ESR2, FAM179B, FTH1, H3F3A, HDAC4, HINFP, HNRNPK, HP1BP3, HRAS, MAX, MCM5, MED6, MGST2, MSH6, PURA, RABGEF1, RBBP6, RBM26, RECQL, RUNX3, SFPQ, SGCB, SMARCA1, SMC1A, SPAG9, UBA2, UBE2B, or USPL1.

In some embodiments, the gene expression response signature comprises an expression level of (1) PKM and SUMO2; and (2) one or more genes selected from: ADAR, ANP32B, ATRX, BRD7, CAPN1, CCDC88A, CFAP206, CGN, CIRBP, CLTC, EEA1, ERICH1, FAM192A, FAM207A, HHEX, KLF3, LCA5, MDC1, MDM2, NFAT5, ARCN1, ARF6, ARNT, ARPC5L, ASB16, ATF7IP, ATP6VOC, BRF1, CHFR, EDA, EFEMP2, ESR2, FAM179B, FTH1, H3F3A, HDAC4, HINFP, HNRNPK, NUCKS1, PML, PNN, PRKAB1, RBCK1, RRP15, SNRPN, TFIP11, THTPA, TMEM87A, TNK2, TPR, TRAPPC4, UBA5, UBE2D1, VPS72, YWHAE, MCM5, MED6, MGST2, MSH6, PURA, RABGEF1, RBBP6, RBM26, RECQL, RUNX3, SFPQ, SGCB, SMARCA1, SMC1A, SPAG9, UBA2, UBE2B, USPL1, HP1BP3, HRAS, or MAX.

In some embodiments, repeated monitoring under time permits or achieves detection of one or more changes in a subject's gene expression profile or characteristics that may impact ongoing treatment regimens. In some embodiments, a change is detected in response to which particular therapy administered to the subject is continued, is altered, or is suspended. In some embodiments, therapy may be altered, for example, by increasing or decreasing frequency and/or amount of administration of one or more agents or treatments with which the subject is already being treated. Alternatively or additionally, in some embodiments, therapy may be altered by addition of therapy with one or more new agents or treatments. In some embodiments, therapy may be altered by suspension or cessation of one or more particular agents or treatments.

To give but one example, if a subject is initially classified as responsive (because the subject's gene expression correlated to a gene expression response signature associated with a disease, disorder, or condition), a given anti-TNF therapy can then be administered. At a given interval (e.g., every six months, every year, etc.), the subject can be tested again to ensure that they still qualify as “responsive” to a given anti-TNF therapy. In the event the gene expression levels for a given subject change over time, and the subject no longer expresses genes associated with the gene expression response signature, or now expresses genes associated with non-responsiveness, the subject's therapy can be altered to suit the change in gene expression.

Accordingly, in some embodiments, the present disclosure provides methods of administering therapy to a subject previously determined not to display a gene expression response signature associated with anti-TNF therapy, wherein the subject does not displays a gene expression response signature associated with response to anti-TNF therapy.

In some embodiments, the present disclosure provides methods of treating subjects with anti-TNF therapy, the method comprising a step of: administering the anti-TNF therapy to subjects who have been determined not to display a gene expression response signature established to distinguish between responsive and non-responsive prior subjects who have received the anti-TNF therapy.

In some embodiments, the present disclosure provides methods further comprising determining, prior to the administering, that a subject does not display the gene expression response signature; and administering the anti-TNF therapy to the subject determined not to display the gene expression response signature.

In some embodiments, the present disclosure provides methods further comprising determining, prior to the administering, that a subject does display the gene expression response signature; and administering a therapy alternative to anti-TNF therapy to the subject determined to display the gene expression response signature.

In some embodiments, the gene expression response signature was established to distinguish between responsive and non-responsive prior subjects who have received the anti-TNF therapy by a method comprising steps of: mapping genes whose expression levels significantly correlate to clinical responsiveness or non-responsiveness to a human interactome map; and selecting a plurality of genes determined to cluster with one another in a human interactome map, thereby establishing the gene expression response signature.

In some embodiments, the gene expression response signature was established to distinguish between responsive and non-responsive prior subjects who have received the anti-TNF therapy by a method comprising steps of: mapping genes whose expression levels significantly correlate to clinical responsiveness or non-responsiveness to a human interactome map; and selecting a plurality of genes determined to be proximal with one another in a human interactome map, thereby establishing the gene expression response signature.

In some embodiments, the present disclosure provides methods further comprising steps of: validating the gene expression response signature by assigning probability of response to a group of known responders and non-responders; and checking the gene expression response signature against a blinded group of responders and non-responders.

In some embodiments, the responsive and non-responsive prior subjects suffered from the same disease, disorder, or condition.

In some embodiments, the subjects to whom the anti-TNF therapy is administered are suffering from the same disease, disorder or condition as the prior responsive and non-responsive prior subjects.

In some embodiments, the gene expression response signature includes expression levels of a plurality of genes derived from a cluster of genes associated with response to anti-TNF therapy on a human interactome map.

In some embodiments, the gene expression response signature includes expression levels of a plurality of genes proximal to genes associated with response to anti-TNF therapy on a human interactome map.

In some embodiments, the gene expression response signature includes expression levels of a plurality of genes determined to cluster with one another in a human interactome map.

In some embodiments, the gene expression response signature includes expression levels of a plurality of genes that are proximal in a human interactome map.

In some embodiments, genes of the subject are measured by at least one of a microarray, RNA sequencing, real-time quantitative reverse transcription PCR (qRT-PCR), bead array, ELISA, and protein expression.

In some embodiments, a disease, disorder, or condition described herein is an autoimmune disease.

In some embodiments, the subject suffers from a disease, disorder, or condition selected from rheumatoid arthritis, psoriatic arthritis, ankylosing spondylitis, Crohn's disease (adult or pediatric), ulcerative colitis, inflammatory bowel disease, chronic psoriasis, plaque psoriasis, hidradenitis suppurativa, asthma, uveitis, juvenile idiopathic arthritis, vitiligo, Graves' ophthalmopathy (also known as thyroid eye disease, or Graves' orbitopathy), and multiple sclerosis.

In some embodiments, the subject suffers from an autoimmune disease selected from rheumatoid arthritis, psoriatic arthritis, ankylosing spondylitis, Crohn's disease (adult or pediatric), ulcerative colitis, inflammatory bowel disease, chronic psoriasis, plaque psoriasis, hidradenitis suppurativa, asthma, uveitis, juvenile idiopathic arthritis, vitiligo, Graves' ophthalmopathy (also known as thyroid eye disease, or Graves' orbitopathy), and multiple sclerosis.

In some embodiments, the anti-TNF therapy is or comprises administration of infliximab, adalimumab, etanercept, certolizumab pegol, golimumab, or biosimilars thereof. In some embodiments, the anti-TNF therapy is or comprises administration of infliximab or adalimumab.

In some embodiments, the present disclosure provides, in a method of administering anti-TNF therapy, the improvement that comprises administering the therapy selectively to subjects who have been determined to display a gene expression response signature established to distinguish between responsive and non-responsive prior subjects who have received the anti-TNF therapy.

In some embodiments, the responsive and non-responsive prior subjects suffered from the same disease, disorder, or condition.

In some embodiments, the subjects to whom the anti-TNF therapy is administered are suffering from the same disease, disorder or condition as the prior responsive and non-responsive prior subjects.

In some embodiments, the gene expression response signature includes expression levels of a plurality of genes derived from a cluster of genes associated with response to anti-TNF therapy on a human interactome map.

In some embodiments, the anti-TNF therapy is or comprises administration of infliximab, adalimumab, etanercept, certolizumab pegol, golimumab, or biosimilars thereof.

In some embodiments, the disease, disorder, or condition is rheumatoid arthritis.

In some embodiments, the disease, disorder, or condition is ulcerative colitis.

In some embodiments, the present disclosure provides use of an anti-TNF therapy in the treatment of a subject determined to display a gene expression response signature established to distinguish between responsive and non-responsive prior subjects who have received the anti-TNF therapy.

In some embodiments, prior to use of the anti-TNF therapy, determining that the subject displays the gene expression response signature. In some embodiments, prior to use of the anti-TNF therapy, determining that the subject does not display the gene expression response signature.

In some embodiments, the gene expression response signature was established to distinguish between responsive and non-responsive prior subjects who have received the anti-TNF therapy by a method comprising steps of: mapping genes whose expression levels significantly correlate to clinical responsiveness or non-responsiveness to a human interactome map; and selecting a plurality of genes determined to cluster with one another in a human interactome map, thereby establishing the gene expression response signature.

In some embodiments, the gene expression response signature was established to distinguish between responsive and non-responsive prior subjects who have received the anti-TNF therapy by a method comprising steps of: mapping genes whose expression levels significantly correlate to clinical responsiveness or non-responsiveness to a human interactome map; and selecting a plurality of genes determined to be proximal with one another in a human interactome map, thereby establishing the gene expression response signature.

In some embodiments, the gene expression response signature was established to distinguish between responsive and non-responsive prior subjects who have received the anti-TNF therapy by the method further comprising steps of validating the gene expression response signature by assigning probability of response to a group of known responders and non-responders; and checking the gene expression response signature against a blinded group of responders and non-responders.

### Systems and Architecture

In some embodiments, the present disclosure provides a method of of validating response to an anti-TNF therapy in a subject, the method comprising: receiving, by a processor of a computing device, a gene expression response signature determined to distinguish between responsive and non-responsive subjects to the anti-TNF therapy; analyzing, by the processor, gene expression of the subject relative to the gene expression response signature to determine whether the subject expresses the gene expression response signature, wherein the gene expression response signature comprising one or more genes selected from: PKM, ADAR, ANP32B, ATRX, BRD7, CAPN1, CCDC88A, CFAP206, CGN, CIRBP, CLTC, EEA1, ERICH1, FAM192A, FAM207A, HHEX, KLF3, LCA5, MDC1, MDM2, NFAT5, ARCN1, ARF6, ARNT, ARPC5L, ASB16, ATF7IP, ATP6VOC, BRF1, CHFR, EDA, EFEMP2, ESR2, FAM179B, FTH1, H3F3A, HDAC4, HINFP, HNRNPK, SUMO2, NUCKS1, PML, PNN, PRKAB1, RBCK1, RRP15, SNRPN, TFIP11, THTPA, TMEM87A, TNK2, TPR, TRAPPC4, UBA5, UBE2D1, VPS72, YWHAE, MCM5, MED6, MGST2, MSH6, PURA, RABGEF1, RBBP6, RBM26, RECQL, RUNX3, SFPQ, SGCB, SMARCA1, SMC1A, SPAG9, UBA2, UBE2B, USPL1, HP1BP3, HRAS, or MAX.

In some embodiments, the present disclosure provides a system for determining or validating responsiveness to anti-TNF therapy for a subject suffering from a disease, the system comprising: a processor of a computing device; and a memory having instructions stored thereon, wherein the instructions, when executed by the processor cause the processor to perform the steps of methods described herein.

In some embodiments, the present disclosure provides a system for determining or validating responsiveness to anti-TNF therapy for a subject suffering from a disease, the system comprising: a processor of a computing device; and a memory having instructions stored thereon, wherein the instructions, when executed by the processor cause the processor to perform the following steps: receiving, by the processor, a gene expression response signature determined to distinguish between responsive and non-responsive subjects to the anti-TNF therapy; analyzing, by the processor, gene expression of the subject relative to the gene expression response signature to determine whether the subject expresses the gene expression response signature, wherein the gene expression response signature comprising one or more genes selected from: PKM, ADAR, ANP32B, ATRX, BRD7, CAPN1, CCDC88A, CFAP206, CGN, CIRBP, CLTC, EEA1, ERICH1, FAM192A, FAM207A, HHEX, KLF3, LCA5, MDC1, MDM2, NFAT5, ARCN1, ARF6, ARNT, ARPC5L, ASB16, ATF7IP, ATP6VOC, BRF1, CHFR, EDA, EFEMP2, ESR2, FAM179B, FTH1, H3F3A, HDAC4, HINFP, HNRNPK, SUMO2, NUCKS1, PML, PNN, PRKAB1, RBCK1, RRP15, SNRPN, TFIP11, THTPA, TMEM87A, TNK2, TPR, TRAPPC4, UBA5, UBE2D1, VPS72, YWHAE, MCM5, MED6, MGST2, MSH6, PURA, RABGEF1, RBBP6, RBM26, RECQL, RUNX3, SFPQ, SGCB, SMARCA1, SMC1A, SPAG9, UBA2, UBE2B, USPL1, HP1BP3, HRAS, or MAX.

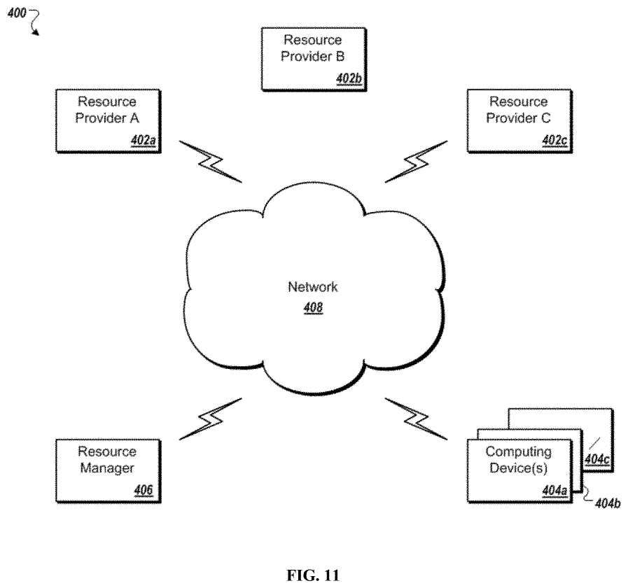

As shown in FIG. 11, an implementation of a network environment 400 for use in providing systems, methods, and architectures as described herein is shown and described. In brief overview, referring now to FIG. 11, a block diagram of an exemplary cloud computing environment 400 is shown and described. The cloud computing environment 400 may include one or more resource providers 402a, 402b, 402c (collectively, 402). Each resource provider 402 may include computing resources. In some implementations, computing resources may include any hardware and/or software used to process data. For example, computing resources may include hardware and/or software capable of executing algorithms, computer programs, and/or computer applications. In some implementations, exemplary computing resources may include application servers and/or databases with storage and retrieval capabilities. Each resource provider 402 may be connected to any other resource provider 402 in the cloud computing environment 400. In some implementations, the resource providers 402 may be connected over a computer network 408. Each resource provider 402 may be connected to one or more computing device 404a, 404b, 404c (collectively, 404), over the computer network 408.

The cloud computing environment 400 may include a resource manager 406. The resource manager 406 may be connected to the resource providers 402 and the computing devices 404 over the computer network 408. In some implementations, the resource manager 406 may facilitate the provision of computing resources by one or more resource providers 402 to one or more computing devices 404. The resource manager 406 may receive a request for a computing resource from a particular computing device 404. The resource manager 406 may identify one or more resource providers 402 capable of providing the computing resource requested by the computing device 404. The resource manager 406 may select a resource provider 402 to provide the computing resource. The resource manager 406 may facilitate a connection between the resource provider 402 and a particular computing device 404. In some implementations, the resource manager 406 may establish a connection between a particular resource provider 402 and a particular computing device 404. In some implementations, the resource manager 406 may redirect a particular computing device 404 to a particular resource provider 402 with the requested computing resource.

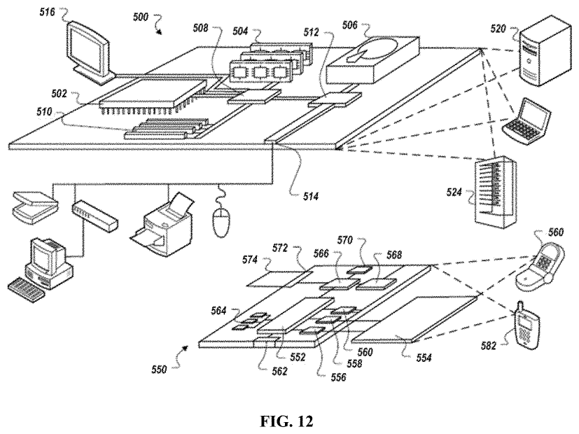

FIG. 12 shows an example of a computing device 500 and a mobile computing device 550 that can be used to implement the techniques described in this disclosure. The computing device 500 is intended to represent various forms of digital computers, such as laptops, desktops, workstations, personal digital assistants, servers, blade servers, mainframes, and other appropriate computers. The mobile computing device 550 is intended to represent various forms of mobile devices, such as personal digital assistants, cellular telephones, smart-phones, and other similar computing devices. The components shown here, their connections and relationships, and their functions, are meant to be examples only, and are not meant to be limiting.

The computing device 500 includes a processor 502, a memory 504, a storage device 506, a high-speed interface 508 connecting to the memory 504 and multiple high-speed expansion ports 510, and a low-speed interface 512 connecting to a low-speed expansion port 514 and the storage device 506. Each of the processor 502, the memory 504, the storage device 506, the high-speed interface 508, the high-speed expansion ports 510, and the low-speed interface 512, are interconnected using various busses, and may be mounted on a common motherboard or in other manners as appropriate. The processor 502 can process instructions for execution within the computing device 500, including instructions stored in the memory 504 or on the storage device 506 to display graphical information for a GUI on an external input/output device, such as a display 516 coupled to the high-speed interface 508. In other implementations, multiple processors and/or multiple buses may be used, as appropriate, along with multiple memories and types of memory. Also, multiple computing devices may be connected, with each device providing portions of the necessary operations (e.g., as a server bank, a group of blade servers, or a multi-processor system). Thus, as the term is used herein, where a plurality of functions are described as being performed by “a processor”, this encompasses embodiments wherein the plurality of functions are performed by any number of processors (one or more) of any number of computing devices (one or more). Furthermore, where a function is described as being performed by “a processor”, this encompasses embodiments wherein the function is performed by any number of processors (one or more) of any number of computing devices (one or more) (e.g., in a distributed computing system).

The memory 504 stores information within the computing device 500. In some implementations, the memory 504 is a volatile memory unit or units. In some implementations, the memory 504 is a non-volatile memory unit or units. The memory 504 may also be another form of computer-readable medium, such as a magnetic or optical disk.

The storage device 506 is capable of providing mass storage for the computing device 500. In some implementations, the storage device 506 may be or contain a computer-readable medium, such as a floppy disk device, a hard disk device, an optical disk device, or a tape device, a flash memory or other similar solid state memory device, or an array of devices, including devices in a storage area network or other configurations. Instructions can be stored in an information carrier. The instructions, when executed by one or more processing devices (for example, processor 502), perform one or more methods, such as those described above. The instructions can also be stored by one or more storage devices such as computer- or machine-readable mediums (for example, the memory 504, the storage device 506, or memory on the processor 502).

The high-speed interface 508 manages bandwidth-intensive operations for the computing device 500, while the low-speed interface 512 manages lower bandwidth-intensive operations. Such allocation of functions is an example only. In some implementations, the high-speed interface 508 is coupled to the memory 504, the display 516 (e.g., through a graphics processor or accelerator), and to the high-speed expansion ports 510, which may accept various expansion cards (not shown). In the implementation, the low-speed interface 512 is coupled to the storage device 506 and the low-speed expansion port 514. The low-speed expansion port 514, which may include various communication ports (e.g., USB, Bluetooth®, Ethernet, wireless Ethernet) may be coupled to one or more input/output devices, such as a keyboard, a pointing device, a scanner, or a networking device such as a switch or router, e.g., through a network adapter.

The computing device 500 may be implemented in a number of different forms, as shown in the figure. For example, it may be implemented as a standard server 520, or multiple times in a group of such servers. In addition, it may be implemented in a personal computer such as a laptop computer 522. It may also be implemented as part of a rack server system 524. Alternatively, components from the computing device 500 may be combined with other components in a mobile device (not shown), such as a mobile computing device 550. Each of such devices may contain one or more of the computing device 500 and the mobile computing device 550, and an entire system may be made up of multiple computing devices communicating with each other.

The mobile computing device 550 includes a processor 552, a memory 564, an input/output device such as a display 554, a communication interface 566, and a transceiver 568, among other components. The mobile computing device 550 may also be provided with a storage device, such as a micro-drive or other device, to provide additional storage. Each of the processor 552, the memory 564, the display 554, the communication interface 566, and the transceiver 568, are interconnected using various buses, and several of the components may be mounted on a common motherboard or in other manners as appropriate.

The processor 552 can execute instructions within the mobile computing device 550, including instructions stored in the memory 564. The processor 552 may be implemented as a chipset of chips that include separate and multiple analog and digital processors. The processor 552 may provide, for example, for coordination of the other components of the mobile computing device 550, such as control of user interfaces, applications run by the mobile computing device 550, and wireless communication by the mobile computing device 550.

The processor 552 may communicate with a user through a control interface 558 and a display interface 556 coupled to the display 554. The display 554 may be, for example, a TFT (Thin-Film-Transistor Liquid Crystal Display) display or an OLED (Organic Light Emitting Diode) display, or other appropriate display technology. The display interface 556 may comprise appropriate circuitry for driving the display 554 to present graphical and other information to a user. The control interface 558 may receive commands from a user and convert them for submission to the processor 552. In addition, an external interface 562 may provide communication with the processor 552, so as to enable near area communication of the mobile computing device 550 with other devices. The external interface 562 may provide, for example, for wired communication in some implementations, or for wireless communication in other implementations, and multiple interfaces may also be used.

The memory 564 stores information within the mobile computing device 550. The memory 564 can be implemented as one or more of a computer-readable medium or media, a volatile memory unit or units, or a non-volatile memory unit or units. An expansion memory 574 may also be provided and connected to the mobile computing device 550 through an expansion interface 572, which may include, for example, a SIMM (Single In Line Memory Module) card interface. The expansion memory 574 may provide extra storage space for the mobile computing device 550, or may also store applications or other information for the mobile computing device 550. Specifically, the expansion memory 574 may include instructions to carry out or supplement the processes described above, and may include secure information also. Thus, for example, the expansion memory 574 may be provide as a security module for the mobile computing device 550, and may be programmed with instructions that permit secure use of the mobile computing device 550. In addition, secure applications may be provided via the SIMM cards, along with additional information, such as placing identifying information on the SIMM card in a non-hackable manner.

The memory may include, for example, flash memory and/or NVRAM memory (non-volatile random access memory), as discussed below. In some implementations, instructions are stored in an information carrier. that the instructions, when executed by one or more processing devices (for example, processor 552), perform one or more methods, such as those described above. The instructions can also be stored by one or more storage devices, such as one or more computer- or machine-readable mediums (for example, the memory 564, the expansion memory 574, or memory on the processor 552). In some implementations, the instructions can be received in a propagated signal, for example, over the transceiver 568 or the external interface 562.

The mobile computing device 550 may communicate wirelessly through the communication interface 566, which may include digital signal processing circuitry where necessary. The communication interface 566 may provide for communications under various modes or protocols, such as GSM voice calls (Global System for Mobile communications), SMS (Short Message Service), EMS (Enhanced Messaging Service), or MMS messaging (Multimedia Messaging Service), CDMA (code division multiple access), TDMA (time division multiple access), PDC (Personal Digital Cellular), WCDMA (Wideband Code Division Multiple Access), CDMA2000, or GPRS (General Packet Radio Service), among others. Such communication may occur, for example, through the transceiver 568 using a radio-frequency. In addition, short-range communication may occur, such as using a Bluetooth®, Wi-Fi™, or other such transceiver (not shown). In addition, a GPS (Global Positioning System) receiver module 570 may provide additional navigation- and location-related wireless data to the mobile computing device 550, which may be used as appropriate by applications running on the mobile computing device 550.

The mobile computing device 550 may also communicate audibly using an audio codec 560, which may receive spoken information from a user and convert it to usable digital information. The audio codec 560 may likewise generate audible sound for a user, such as through a speaker, e.g., in a handset of the mobile computing device 550. Such sound may include sound from voice telephone calls, may include recorded sound (e.g., voice messages, music files, etc.) and may also include sound generated by applications operating on the mobile computing device 550.

The mobile computing device 550 may be implemented in a number of different forms, as shown in the figure. For example, it may be implemented as a cellular telephone 580. It may also be implemented as part of a smart-phone 582, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term machine-readable signal refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, the modules described herein can be separated, combined or incorporated into single or combined modules. The modules depicted in the figures are not intended to limit the systems described herein to the software architectures shown therein.

Elements of different implementations described herein may be combined to form other implementations not specifically set forth above. Elements may be left out of the processes, computer programs, databases, etc. described herein without adversely affecting their operation. In addition, the logic flows depicted in the figures do not require the particular order shown, or sequential order, to achieve desirable results. Various separate elements may be combined into one or more individual elements to perform the functions described herein. In view of the structure, functions and apparatus of the systems and methods described here, in some implementations.

It is contemplated that systems, architectures, devices, methods, and processes of the claimed invention encompass variations and adaptations developed using information from the embodiments described herein. Adaptation and/or modification of the systems, architectures, devices, methods, and processes described herein may be performed, as contemplated by this description.

Throughout the description, where articles, devices, systems, and architectures are described as having, including, or comprising specific components, or where processes and methods are described as having, including, or comprising specific steps, it is contemplated that, additionally, there are articles, devices, systems, and architectures of the present invention that consist essentially of, or consist of, the recited components, and that there are processes and methods according to the present invention that consist essentially of, or consist of, the recited processing steps.

It should be understood that the order of steps or order for performing certain action is immaterial so long as the invention remains operable. Moreover, two or more steps or actions may be conducted simultaneously.

The mention herein of any publication, for example, in the Background section, is not an admission that the publication serves as prior art with respect to any of the claims presented herein. The Background section is presented for purposes of clarity and is not meant as a description of prior art with respect to any claim.

Headers are provided for the convenience of the reader—the presence and/or placement of a header is not intended to limit the scope of the subject matter described herein.

### EMBODIMENTS

The following exemplary embodiments are intended to be non-limiting examples of particular aspects of the disclosure.
1. A method of treating a subject suffering from a disease, disorder, or condition with an anti-TNF therapy, the method comprising a step of:
   administering the anti-TNF therapy to subjects who have been determined not to display a gene expression response signature;
   wherein the gene expression response signature has been derived by analysis of gene expression levels in biological samples from subjects who have previously received the anti-TNF therapy (“prior subjects”) and have been determined to respond or not to respond to the anti-TNF therapy; and
   wherein the gene expression response signature comprises an expression level of PKM and SUMO2.
2. The method of Embodiment 1, wherein the gene expression response signature comprises an expression level of one or more genes selected from: ADAR, ANP32B, ATRX, BRD7, CAPN1, CCDC88A, CFAP206, CGN, CIRBP, CLTC, EEA1, ERICH1, FAM192A, FAM207A, HHEX, KLF3, LCA5, MDC1, MDM2, NFAT5, NUCKS1, PML, PNN, PRKAB1, RBCK1, RRP15, SNRPN, TFIP11, THTPA, TMEM87A, TNK2, TPR, TRAPPC4, UBA5, UBE2D1, VPS72, or YWHAE.
3. The method of Embodiment 1, wherein the gene expression response signature comprises an expression level of one or more genes selected from: ARCN1, ARF6, ARNT, ARPC5L, ASB16, ATF7IP, ATP6VOC, BRF1, CHFR, EDA, EFEMP2, ESR2, FAM179B, FTH1, H3F3A, HDAC4, HINFP, HNRNPK, HP1BP3, HRAS, MAX, MCM5, MED6, MGST2, MSH6, PURA, RABGEF1, RBBP6, RBM26, RECQL, RUNX3, SFPQ, SGCB, SMARCA1, SMC1A, SPAG9, UBA2, UBE2B, or USPL1.
4. The method of Embodiment 1, wherein the gene expression response signature comprises an expression level of one or more genes selected from: ADAR, ANP32B, ATRX, BRD7, CAPN1, CCDC88A, CFAP206, CGN, CIRBP, CLTC, EEA1, ERICH1, FAM192A, FAM207A, HHEX, KLF3, LCA5, MDC1, MDM2, NFAT5, ARCN1, ARF6, ARNT, ARPC5L, ASB16, ATF7IP, ATP6VOC, BRF1, CHFR, EDA, EFEMP2, ESR2, FAM179B, FTH1, H3F3A, HDAC4, HINFP, HNRNPK, NUCKS1, PML, PNN, PRKAB1, RBCK1, RRP15, SNRPN, TFIP11, THTPA, TMEM87A, TNK2, TPR, TRAPPC4, UBA5, UBE2D1, VPS72, YWHAE, MCM5, MED6, MGST2, MSH6, PURA, RABGEF1, RBBP6, RBM26, RECQL, RUNX3, SFPQ, SGCB, SMARCA1, SMC1A, SPAG9, UBA2, UBE2B, USPL1, HP1BP3, HRAS, or MAX.
5. The method of any one of Embodiments 1-3, wherein the step of administering comprises a administering the anti-TNF therapy to patients who do not display either of a first or second gene expression signature, wherein:
   the first gene expression signature comprises or consists of expression levels for one or more, or each of: ADAR, ANP32B, ATRX, BRD7, CAPN1, CCDC88A, CFAP206, CGN, CIRBP, CLTC, EEA1, ERICH1, FAM192A, FAM207A, HHEX, KLF3, LCA5, MDC1, MDM2, NFAT5, NUCKS1, PML, PNN, PRKAB1, RBCK1, RRP15, SNRPN, TFIP11, THTPA, TMEM87A, TNK2, TPR, TRAPPC4, UBA5, UBE2D1, VPS72, YWHAE, SUMO2, or PKM.
   the second gene expression signature comprises or consists of expression levels for one or more, or each of: ARCN1, ARF6, ARNT, ARPCSL, ASB16, ATF7IP, ATP6VOC, BRF1, CHFR, EDA, EFEMP2, ESR2, FAM179B, FTH1, H3F3A, HDAC4, HINFP, HNRNPK, HP1BP3, HRAS, MAX, MCM5, MED6, MGST2, MSH6, PURA, RABGEF1, RBBP6, RBM26, RECQL, RUNX3, SFPQ, SGCB, SMARCA1, SMC1A, SPAG9, UBA2, UBE2B, USPL1, SUMO2, or PKM.
6. The method of any one of Embodiments 1-5, wherein the anti-TNF therapy is or comprises administration of infliximab, adalimumab, etanercept, certolizumab pegol, golimumab, or biosimilars thereof
7 The method of any one of Embodiments 1-6, wherein the anti-TNF therapy is or comprises administration of infliximab or adalimumab.
8. The method of any one of Embodiments 1-7, wherein the anti-TNF therapy is or comprises infliximab.
9. The method of any one of Embodiments 1-8, further comprising:
   determining, prior to the administering, that a subject does not display the gene expression response signature; and
   administering the anti-TNF therapy to the subject determined not to display the gene expression response signature.
10. The method of any one of Embodiments 1-8, further comprising:
   determining, prior to the administering, that a subject does display the gene expression response signature; and
   administering a therapy alternative to anti-TNF therapy to the subject determined to display the gene expression response signature.
11. The method of Embodiment 10, wherein the therapy alternative to anti-TNF therapy is selected from rituximab, sarilumab, tofacitinib citrate, leflunomide, vedolizumab, tocilizumab, anakinra, and abatacept.
12. The method of Embodiments 10 or 11, wherein the step of determining comprises measuring gene expression by at least one of a microarray, RNA sequencing, real-time quantitative reverse transcription PCR (qRT-PCR), bead array, and ELISA.
13. The method of any one of Embodiments 1-12, wherein the gene expression response signature distinguishes 65% of prior subjects that are responsive to the anti-TNF therapy.
14. The method of any one of Embodiments 1-13, wherein the gene expression response signature distinguishes 70% of prior subjects that are responsive to the anti-TNF therapy.
15. The method of any one of Embodiments 1-14, wherein the gene expression response signature distinguishes 80% of prior subjects that are responsive to the anti-TNF therapy.
16. The method of any one of Embodiments 1-15, wherein the gene expression response signature distinguishes 90% of prior subjects that are responsive to the anti-TNF therapy.
17. The method of any one of Embodiments 1-16, wherein the gene expression response signature distinguishes 100% of prior subjects that are responsive to the anti-TNF therapy.
18. The method of any one of Embodiments 1-17, wherein the gene expression response signature distinguishes 65% of prior subjects that are non-responsive to the anti-TNF therapy.
19. The method of any one of Embodiments 1-18, wherein the gene expression response signature distinguishes 70% of prior subjects that are non-responsive to the anti-TNF therapy.
20. The method of any one of Embodiments 1-19, wherein the gene expression response signature distinguishes 80% of prior subjects that are non-responsive to the anti-TNF therapy.

21. The method of any one of Embodiments 1-20, wherein the gene expression response signature distinguishes 90% of prior subjects that are non-responsive to the anti-TNF therapy.
22. The method of any one of Embodiments 1-21, wherein the gene expression response signature distinguishes 100% of prior subjects that are non-responsive to the anti-TNF therapy.
23. The method of any one of Embodiments 1-22, wherein the disease, disorder, or condition is selected from rheumatoid arthritis, psoriatic arthritis, ankylosing spondylitis, Crohn's disease (adult or pediatric), ulcerative colitis, inflammatory bowel disease, chronic psoriasis, plaque psoriasis, hidradenitis suppurativa, asthma, uveitis, juvenile idiopathic arthritis, vitiligo, Graves' ophthalmopathy (also known as thyroid eye disease, or Graves' orbitopathy), and multiple sclerosis.
24. The method of Embodiment 23, wherein the disease, disorder, or condition is ulcerative colitis.
25. A kit comprising a gene expression response signature established to distinguish between responsive and non-responsive prior subjects suffering from a disease, disorder, or condition and who have received anti-TNF therapy, wherein the gene expression response signature comprises an expression level of PKM and SUMO2.
26. The kit of Embodiment 25, wherein the gene expression response signature comprises an expression level of one or more genes selected from: ADAR, ANP32B, ATRX, BRD7, CAPN1, CCDC88A, CFAP206, CGN, CIRBP, CLTC, EEA1, ERICH1, FAM192A, FAM207A, HHEX, KLF3, LCA5, MDC1, MDM2, NFAT5, NUCKS1, PML, PNN, PRKAB1, RBCK1, RRP15, SNRPN, TFIP11, THTPA, TMEM87A, TNK2, TPR, TRAPPC4, UBA5, UBE2D1, VPS72, or YWHAE.
27. The kit of Embodiment 25, wherein the gene expression response signature comprises an expression level one or more genes selected from: ARCN1, ARF6, ARNT, ARPCSL, ASB16, ATF7IP, ATP6VOC, BRF1, CHFR, EDA, EFEMP2, ESR2, FAM179B, FTH1, H3F3A, HDAC4, HINFP, HNRNPK, HP1BP3, HRAS, MAX, MCM5, MED6, MGST2, MSH6, PURA, RABGEF1, RBBP6, RBM26, RECQL, RUNX3, SFPQ, SGCB, SMARCA1, SMC1A, SPAG9, UBA2, UBE2B, or USPL1.
28. The kit of Embodiment 25, wherein the gene expression response signature comprises an expression level of one or more genes selected from: ADAR, ANP32B, ATRX, BRD7, CAPN1, CCDC88A, CFAP206, CGN, CIRBP, CLTC, EEA1, ERICH1, FAM192A, FAM207A, HHEX, KLF3, LCA5, MDC1, MDM2, NFAT5, ARCN1, ARF6, ARNT, ARPCSL, ASB16, ATF7IP, ATP6VOC, BRF1, CHFR, EDA, EFEMP2, ESR2, FAM179B, FTH1, H3F3A, HDAC4, HINFP, HNRNPK, NUCKS1, PML, PNN, PRKAB1, RBCK1, RRP15, SNRPN, TFIP11, THTPA, TMEM87A, TNK2, TPR, TRAPPC4, UBA5, UBE2D1, VPS72, YWHAE, MCM5, MED6, MGST2, MSH6, PURA, RABGEF1, RBBP6, RBM26, RECQL, RUNX3, SFPQ, SGCB, SMARCA1, SMC1A, SPAG9, UBA2, UBE2B, USPL1, HP1BP3, HRAS, or MAX.
29. The kit of any one of Embodiments 25-28, wherein the gene expression response signature comprises a first or second gene expression signature, wherein:
   the first gene expression signature comprises expression levels for one or more, or each of: ADAR, ANP32B, ATRX, BRD7, CAPN1, CCDC88A, CFAP206, CGN, CIRBP, CLTC, EEA1, ERICH1, FAM192A, FAM207A, HHEX, KLF3, LCA5, MDC1, MDM2, NFAT5, NUCKS1, PML, PNN, PRKAB1, RBCK1, RRP15, SNRPN, TFIP11, THTPA, TMEM87A, TNK2, TPR, TRAPPC4, UBA5, UBE2D1, VPS72, YWHAE, SUMO2, or PKM.
   the second gene expression signature comprises expression levels for one or more, or each of: ARCN1, ARF6, ARNT, ARPCSL, ASB16, ATF7IP, ATP6VOC, BRF1, CHFR, EDA, EFEMP2, ESR2, FAM179B, FTH1, H3F3A, HDAC4, HINFP, HNRNPK, HP1BP3, HRAS, MAX, MCM5, MED6, MGST2, MSH6, PURA, RABGEF1, RBBP6, RBM26, RECQL, RUNX3, SFPQ, SGCB, SMARCA1, SMC1A, SPAG9, UBA2, UBE2B, USPL1, SUMO2, or PKM.
30. The kit of any one of Embodiments 25-29, wherein the disease, disorder, or condition is selected from rheumatoid arthritis, psoriatic arthritis, ankylosing spondylitis, Crohn's disease (adult or pediatric), ulcerative colitis, inflammatory bowel disease, chronic psoriasis, plaque psoriasis, hidradenitis suppurativa, asthma, uveitis, juvenile idiopathic arthritis, vitiligo, Graves' ophthalmopathy (also known as thyroid eye disease, or Graves' orbitopathy), and multiple sclerosis.
31. The kit of any one of Embodiments 25-30, wherein the disease, disorder, or condition is ulcerative colitis, inflammatory bowel disease, or Crohn's disease.
32. The kit of any one of Embodiments 25-31, wherein the disease, disorder, or condition is ulcerative colitis.
33. The kit of any one of Embodiments 25-32, wherein the anti-TNF therapy is or comprises administration of infliximab, adalimumab, etanercept, certolizumab pegol, golimumab, or biosimilars thereof.
34. The kit of any one of Embodiments 25-33, wherein the anti-TNF therapy is or comprises administration of infliximab or adalimumab.
35. The kit of any one of Embodiments 25-34, wherein the anti-TNF therapy is or comprises administration of infliximab.
36. The kit of any one of Embodiments 25-35, wherein the kit compares levels of gene expression of a subject to the gene expression response signature established to distinguish between responsive and non-responsive prior subjects who have received anti-TNF therapy.
37. The kit of any one of Embodiments 25-36, wherein the levels of gene expression of the subject are measured by at least one of a microarray, RNA sequencing, real-time quantitative reverse transcription PCR (qRT-PCR), bead array, and ELISA.
38. The kit of any one of Embodiments 25-37, wherein the levels of gene expression of the subject are measured by RNA sequencing.
39. In a method of administering anti-TNF therapy to a subject suffering from a disease, disorder, or condition, the improvement that comprises administering the anti-TNF therapy to subjects who have been determined not to display a gene expression response signature established to distinguish between responsive and non-responsive prior subjects who have received the anti-TNF therapy, wherein the gene expression response signature comprises an expression level of PKM and SUMO2.
40. A method for treating a patient suffering from a disease, disorder or condition with anti-TNF therapy, the method comprising the steps of:
   determining whether the patient is a likely responder to anti-TNF therapy by:
      obtaining or having obtained a biological sample from the patient; and
      performing or having performed an assay on the biological sample to determine if the patient displays a particular gene expression response signature, wherein the gene expression response signature has been derived by analysis of gene expression levels in biological samples from subjects who have previously received the anti-TNF therapy (“prior subjects”) and have been determined to respond or not to respond to the anti-TNF therapy; and
      if the performing determines that the patient is a likely responder, then administering the anti-TNF therapy; and
      if the performing determines that the patient is a likely non-responder, then administering an alternative therapy.
41. The method of Embodiment 40, wherein the performing determines that the subject is a likely non-responder if the subject displays a gene expression response signature determined to correlate with non-responsiveness.
42. The method of Embodiment 40, wherein the performing determines that the subject is a likely non-responder if the subject does not display a gene expression response signature determined to correlate with responsiveness.
43. The method of Embodiment 40, wherein the performing determines that the subject is a likely responder if the subject displays a gene expression response signature determined to correlate with responsiveness.
44. The method of Embodiment 40, wherein the performing determines that the subject is a likely responder if the subject does not display a gene expression response signature determined to correlate with non-responsiveness.
45. A method of treating subjects suffering from an inflammatory disorder with an alternative to anti-TNF therapy, the method comprising a step of:
   administering the alternative to anti-TNF therapy to subjects who have been determined to display a particular gene expression response signature,
   wherein the gene expression response signature has been derived by retrospective analysis of gene expression levels in biological samples from subjects who have previously received the anti-TNF therapy (“prior subjects”) and have been determined to respond or not to respond to anti-TNF therapy.

### EXAMPLES

Examples below demonstrate gene expression response signatures (otherwise referred to as “classifiers” below) characteristic of subjects who do or do not respond to anti-TNF therapy.

### Example 1: Determining Responder and Non-Responder Patient Populations—Ulcerative Colitis

In accordance with the present disclosure, gene expression data from subjects diagnosed with ulcerative colitis (UC) who had received anti-TNF therapy was used to determine patients who are responders and non-responders to anti-TNF therapy. This UC cohort (GSE12251) included 23 patients diagnosed with UC, 11 of which did not respond to anti-TNF-therapy. The gene expression data for this cohort were generated using the Affymetrix® platform.

The gene expression data was analyzed define a set of genes (response signature genes) whose expression patterns distinguish responders and non-responders. To do this, genes with significant gene expression deviations between responders and non-responders were relied on. Unlike conventional differential expression methods that look for high fold changes in gene expression between two groups, the present disclosure provides the insight that small but significant changes between two groups of patients should be included. The present disclosure thus identifies the source of a problem with conventional differential expression technologies.

Without wishing to be bound by any particular theory, the present disclosure provides an insight that small but significant differences impact responsiveness to therapy. Indeed, the present disclosure notes that, given that patients in these cohorts are all diagnosed with the same disease, they often may not manifest big FCs across genes. The present disclosure demonstrates that even very small but significant changes in gene expression will lead to a different treatment outcome.

Additionally, the present disclosure demonstrates that analysis of genes displaying small (but significant) expression differences, in context of a human “interactome” map, defines signatures that reliably distinguish responders from non-responders.

### In-cohort Analysis

Using a human interactome (“HI”) map of gene connectivity that reveals features of underlying biology of response and is useful for understanding response signature genes.

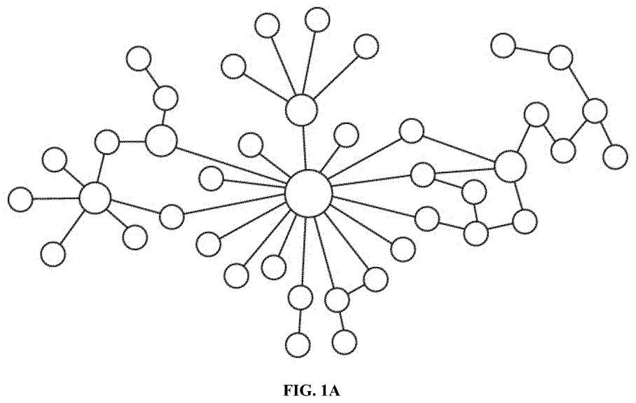

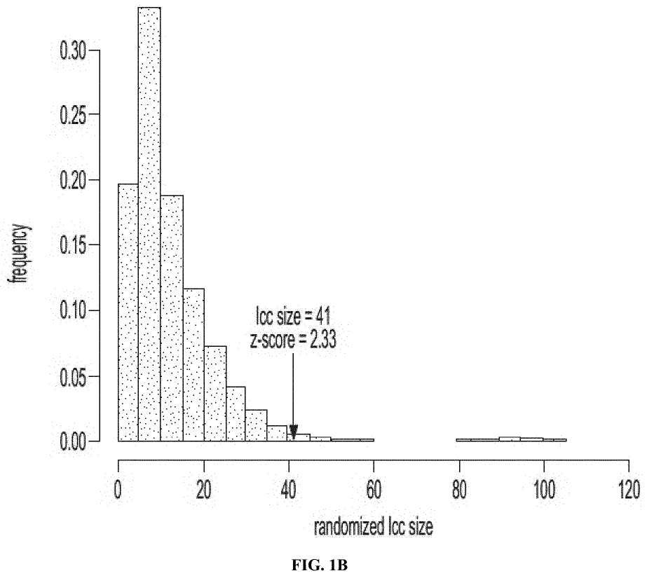

The top 200 genes (as measured by p-value from lowest to highest) whose expression values across patients were significantly correlated to clinical outcome after treatment were selected and mapped to HI. It was observed that even though these genes have been found using the gene expression data only, they form a significant cluster (module) on the HI, with the large connected component (“LCC,” i.e., classifier genes) being much bigger that what is expected by chance HI (FIGS. 1A and 1B). Existence of such significant modules (z-score>1.6) has been repeatedly shown to be associated with underlying disease biology. See Barabási, et al., “Network medicine: a network-based approach to human disease,” Nat. Rev. Genet, 12(1):56-68 (January 2011); Hall et al, “Genetics and the placebo effect: the placebome,” Trends Mol. Med., 21(5):285-294 (May 2015); del Sol, et al., “Diseases as network perturbations,” Curr. Opin. Biotechnol., 21(4):566-571 (August 2010).

FIGS. 1A and 1B show the subnetwork containing the genes correlated to phenotypic outcome in UC cohort as well as their interactions. A significant number of genes found by gene expression analysis form the LCC of the subgraph. The LCC genes (classifier genes) were then utilized to feed and train a probabilistic neural network. The result of the analysis shows a near perfect classifier with an Area Under the Curve (AUC) of 0.98 and with 100% accuracy in predicting non-responders.

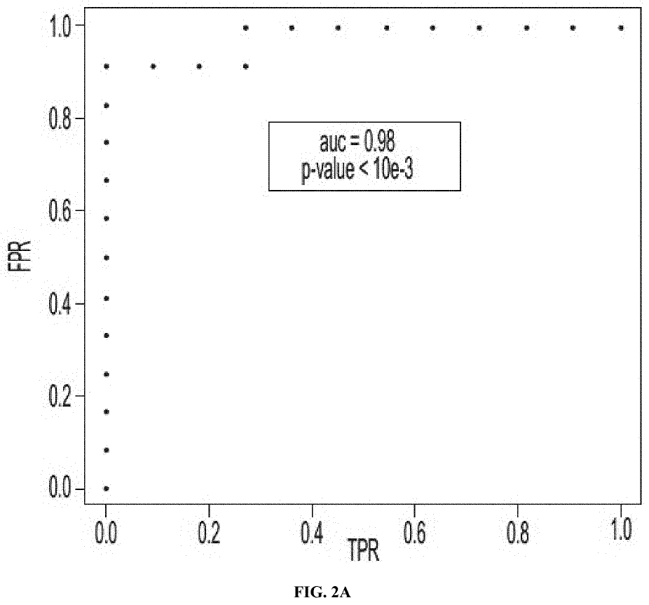

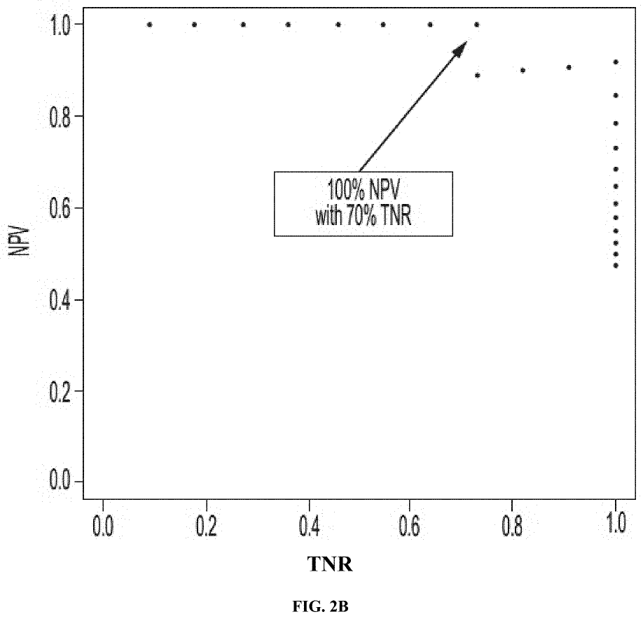

The performance of trained classifiers was validated using a leave-one-out cross validation approach. FIGS. 2A and 2B show the receiver operator curves (ROC) as well as negative prediction power (predicting non-responders) of the classifier. The classifier is able to detect 70% of the non-responders within a cohort.

TABLE 2

| | No. Genes | No. Genes | | |
|---|---|---|---|---|
| Cohort ID | Selected | in HI | LCC Size | Significance |
| GSE12251 | 200 | 193 | 41 | 2.33 |

Table 2 represents the number and topological properties (i.e., the size of the largest component on the network and its significance) of response signature genes when mapped onto the network.

A known and major drawback of traditional gene expression analysis is the inability to reproduce the results across different studies. See loannidis J. P. A., “Why most published research findings are false,” PLoS Med. 2(8):e124 (2005); Goodman S. N., et al., “What does research reproducibility mean?” Sci. Transl. Med., 8(341):341-353 (2016); Ioannidis J. P., et al. “Replication validity of genetic association studies.” Nat. Genet. 29:(3)306-309 (Nov. 2001). Below, it is shown that the methods and systems described herein are able to make high accuracy predictions across cohorts. To estimate the power of the classifier, the classifier is tested in a completely independent cohort (GSE14580) and in a blinded fashion. The independent UC cohort includes 16 non-responders and 8 responders.

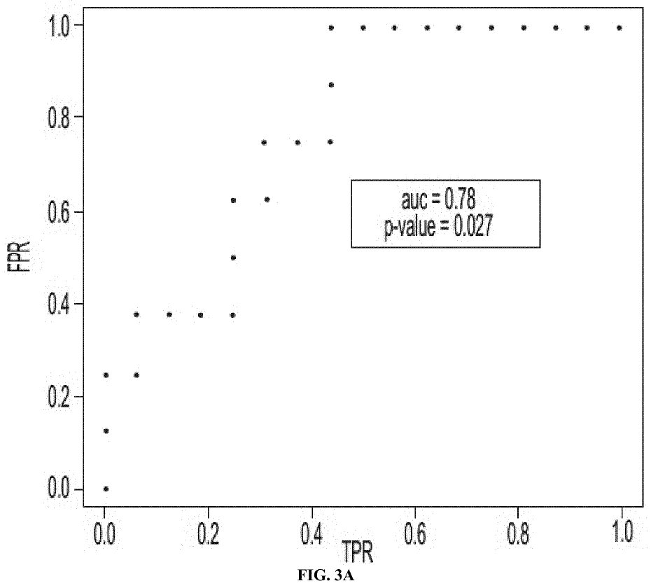

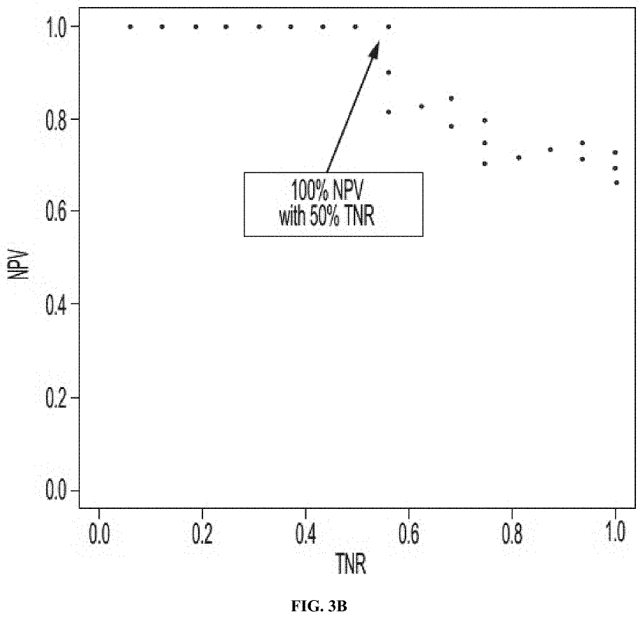

For cross-platform validation, the two cohorts were merged and batch effects removed using the R package, ComBat, a tool used for batch-adjusting gene expression data. See Johnson W. E., et al., “Adjusting batch effects in microarray expression data using empirical Bayes methods,” Biostatistics 8(1), 118-127 (2007). The performance of the designed classifier was tested in the independent cohort (leave-one-batch-out cross validation). FIGS. 3A and 3B show the ROC and negative prediction curves associated with cross-cohort performance of the designed classifier. The trained classifier shows significantly high performance in the independent cohort with AUC of 0.78.

Aside from the high cross-cohort performance assessed by AUC, cross-cohort NPV (Negative Predictive Value) and TNR (True Negative Rate), which indicates the accuracy of detecting non-responders in a blind cohort, were also estimated (FIG. 3B). The cross-cohort validation shows that the classifier is able to predict at least 50% of non-responders (NPV=1, TNR=0.5). The classifier is able to detect more non-responders (TNR>0.5), which results in slight drop in NPV (FIG. 3B). Nevertheless, regardless of the selected point on the curve, the classifier meets or exceeds the commercial criteria (NPV of 0.9 and TNR of 0.5) set by health insurance companies.

### Disease Biology of Non-Responders

The network defined by the analysis described herein provides insights into underlying biology of this response prediction. The classifier genes within the response module were analyzed using GO terms to identify the most highly enriched pathways. We found that inflammatory signaling pathways (including TNF signaling) were highly enriched, as were pathways linked to sumoylation, ubiquitination, proteasome function, proteolytic degradation and antigen presentation in immune cells. Thus, the network approach described herein has captured protein interactions for selecting genes within the response module that clearly reflect the biology of the disease and drug response at the independent patient level and allow the accurate prediction of response to anti-TNF therapies from a baseline sample.

### DISCUSSION

A known and significant problem with existing anti-TNF therapy approaches is that “many patients do not respond to the . . . therapy (primary non-response—PNR), or lose response during the treatment (secondary loss of response—LOR).” See, e.g., Roda et al., Clin Gastroentorl. 7:e135, January 2016. Specifically, reports indicate that “around 10-30% of patients do not respond to the initial treatment and 23-46% of patients lose response over time” Id. Thus, overall, the drug response rate for anti-TNF therapy (and in particular for anti-TNF therapy to treat UC patients) is below 65%, resulting in continued disease progression and escalating treatment costs for the majority of the patient population. Moreover, billions of dollars are spent prescribing anti-TNF therapies to patients that don't respond. There is a significant need for development of a technology that can identify responder vs non-responder subjects, prior to initiation of therapy, at the time that therapy (e.g., a particular dose) is administered, and/or over time as therapy has been or is received.

Gene expression data has been touted as holding the promise of being able to uncover disease biology of individual patients in complex diseases, but up until now the data has been difficult to interpret, and efforts to develop biomarkers (e.g., expression signatures) for therapeutic responsiveness have failed in cross-cohort validation tests. The present disclosure provides new technologies that, for example, consider relatively small changes in expression levels and/or participation of genes in relevant parts of the human interactome.

As already noted, the present disclosure demonstrates that projecting baseline gene expression profiles from UC patients that are non-responders to anti-TNF therapy on the HI, reveals that such profiles cluster and form a large connected module that describes the non-responders' disease biology. In accordance with the present invention, a classifier developed from genes expressed in this module predicts non-response with a high level of accuracy and has been validated in a completely independent cohort (cross-cohort validation). Furthermore, this classifier meets the commercial criteria set by insurance companies and is therefore ready for clinical development and future commercialization.

### Methods

Microarray Analysis

Cohort 1, GSE14580: Twenty-four patients with active UC, refractory to corticosteroids and/or immunosuppression, underwent colonoscopy with biopsies from diseased colon within a week prior to the first intravenous infusion of 5 mg infliximab per kg body weight. Response to infliximab was defined as endoscopic and histologic healing at 4-6 weeks after first infliximab treatment using the MAYO score. Six control patients with normal colonoscopy were included. Total RNA was isolated from colonic mucosal biopsies, labelled and hybridized to Affymetrix® Human Genome U133 Plus 2.0 Arrays.

Cohort 2, GSE12251: Twenty-two patients underwent colonoscopy with biopsy before infliximab treatment. Response to infliximab was defined as endoscopic and histologic healing at week 8 using the MAYO score (P2, 5, 9, 10, 14, 15, 16, 17, 24, 27, 36, and 45 as responders; P3, 12, 13, 19, 28, 29, 32, 33, 34, and 47 as non-responders). Messenger RNA was isolated from pre-infliximab biopsies, labeled and hybridized to Affymetrix® HGU133 Plus_2.0 Array.

Identification of Classifier Genes

Genes with expression values across patients that were significantly correlated to clinical measures after treatment were selected as best determinants of response. These genes were mapped on the consolidated Human Interactome (“HI”). The consolidated Human Interactome collects physical protein interactions between a cell's molecular components relying on experimental support. The material reported by Barabási et al. in “Uncovering disease-disease relationships through the incomplete interactome,” Science, 347(6224):1257601 (February 2015), the entirety of which is incorporated herein by reference, provides instruction regarding how to build and curate a Human Interactome. The genes on the Human Interactome are not randomly scattered on the network. Instead, they significantly interact with each other, reflecting the existence of an underlying disease biology module that explains response.

Human Interactome

As noted, the HI contains experimentally supported physical interactions between cellular components. These interactions were queried from several resources but only selected those that are supported by experimental validation. Most of the interactions in the HI are from unbiased high-throughput studies such as Y2H. All included data were experimentally supported interactions that have been reported in at least two publications. These interactions include, regulatory, metabolic, signaling and binary interactions. The HI contains about 17 k cellular components and over 200K interactions among them. Unlike other interaction databases, no computationally inferred interaction were included, nor any interaction curated from text parsing of literature with no experimental validation.

Classifier Design and Validation

Genes identified above were used as features of a probabilistic neural network. The classifier was validated using leave-one-out and/or k-fold cross validation within a given cohort. The classifier was trained based on the outcome data provided on all patients but the one left out. The classifier was blind to the response outcome of that left out patient. Predicting the outcome of the patient that has been left out then validated the trained classifier. This procedure was repeated so that each patient was left out once. The classifier provided a probability for each patient reflecting whether they belong to responder or non-responder group. The logarithm of likelihood ratio was used to assign a score to each patient. Patients were then ranked based on their score and prediction accuracy values were estimated by varying the classifier threshold resulting in the ROC curves. In particular, each patient is given a score by the trained classifier. The prediction accuracy is measured for the entire cohort as a whole and by checking whether given scores across patients well distinguish responders and non-responders. Prediction performance is generally measured by the Area Under the Curve (AUC). When higher levels of accuracy are required, negative predictive value (NPV) and true negative rate (TNR) can be used. The score cutoff that results in best group separation (e.g., highest NPV) is set for future predictions.

### Example 2: Determining Responder and Non-Responder Patient Populations—Rheumatoid Arthritis

Analogous to Example 1, the present Example 2 describes prediction of response and/or non-response to anti-TNF therapy in patients suffering from rheumatoid arthritis (RA). The presently described predictions satisfy the performance threshold identified by payers and physicians of Negative Predictive Value (NPV) of 0.9 and True Negative Rate (TNR) of 0.5.

In the present example, gene expression data from baseline blood samples for two cohorts comprising a total of 89 RA patients were analyzed. The methodology utilized in the present Example to develop a classifier (i.e., a gene expression response signature) that predicted response and/or non-response to anti-TNF therapy included a four step process. First, initial genes were selected based on differential expression between responders and non-responders to anti-TNF therapy. Second, such genes were projected on the human interactome to determine which genes form a significant and biologically relevant cluster. Third, genes that cluster on the interactome were selected and fed into a probabilistic neural network (PNN) to develop the final classifiers. And fourth, each classifier was validated using leave-one-out validation in the training set, and validated cross-cohort in an independent cohort of patients (test set). For RA, the final classifier contained 9 genes and reached an NPV of 0.91 and TNR of 0.67 in the test set.

The developed classifiers meet the performance thresholds set by payers and physicians; those skilled in the art will appreciate that these classifiers are useful tests that predict non-response to anti-TNFs prior to initiation of therapy and/or to assess desirability of altering administered therapy. Among other things, provided technology therefore permits selection of therapy (whether initial therapy or continued or altered therapy), including enabling patients to be switched onto alternative therapies faster, resulting in substantial clinical benefits to patients and savings to the healthcare system.

### Data Description

The response prediction analysis in RA utilized in the present Example was based on two individual cohorts (Tables 3 and 4). Response was measured 14-weeks after initiation of anti-TNF therapy, with response rates (Good responders; DAS28 improvement>1.2, corresponding to LDA or remission) in cohort 1 and 2 of 30% and 23%, respectively. Cohort 1 was used to train the classifier and cohort 2 was used as the independent test cohort to validate the predictive power of the classifier.

The analyses were conducted on RNA expression data generated from whole blood, before initiation of therapy, using an Illumina® BeadArray platform and provided as standard output of BeadStudio. Raw data was normalized and processed using lumi package in R.

TABLE 3

| Clinical response according | | |
|---|---|---|
| to EULAR DAS28 criteria | Cohort 1 | Cohort 2 |
| No. Good responders | 15 | 9 |
| No. Moderate responders | 15 | 15 |
| No. Non-responders | 20 | 15 |

TABLE 4

| | DAS28 Improvement | | |
|---|---|---|---|
| Baseline DAS28 | >1.2 | >0.6 & ≤1.2 | ≤0.6 |
| ≤3.2 | Good | Moderate | No response |
| | response | response | |
| >3.2 & ≤5.1 | Moderate | Moderate | No response |
| | response | response | |
| >5.1 | Moderate | No response | No response |
| | response | | |

### Identifying Classifier Genes

Expression values for over 10,000 probes (genes) were available in each patient; those skilled in the art will appreciate the challenges associated with defining a set of genes (features) that effectively distinguishes response from such a volume of data. Insights provided by the present disclosure, including that particularly useful genes for inclusion in a classifier may, in some embodiments, be those with relatively small changes, permit effective selection of gene (feature) set(s) for use in a classifier.

In the present Example, genes for inclusion in an RA classifier were selected via a multi-step analysis: First, genes were ranked based on their significance of correlation to patient's response outcome (change in baseline DAS28 score at week 14) using Pearson correlation resulting in 200 top ranked genes (Feature set 1). Unlike conventional differential expression methods that look for highest fold changes in gene expression between two groups, the present Example captures small but significant changes between two groups of patients.

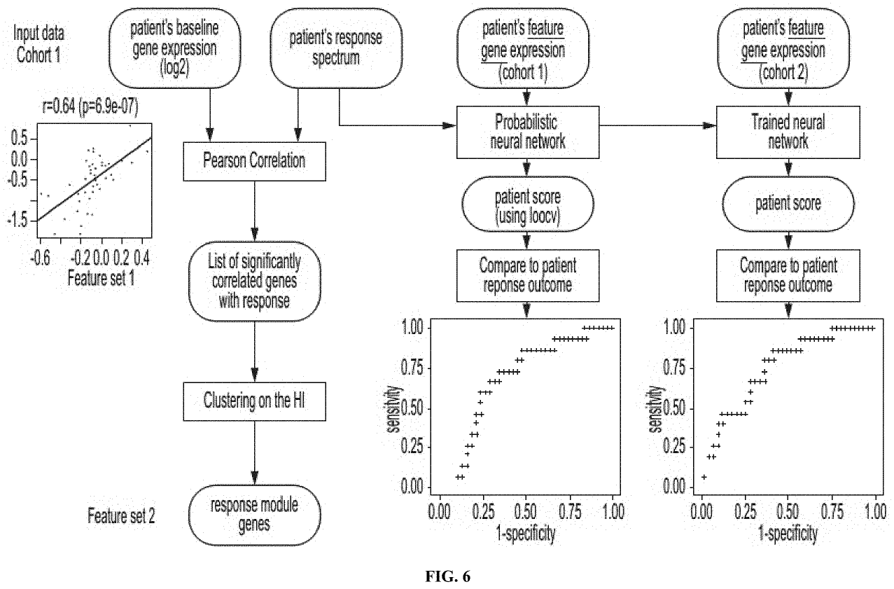

Second, the present disclosure appreciates that gene products (proteins) do not function in isolation, and furthermore appreciates that reference to the interactome—a map of protein interconnectivity—can valuably be used as a blueprint to understand roles played by individual gene products in context (i.e., in biology of cells and/or organisms). By mapping the 200 genes identified above on the interactome, a significant cluster, or response module, consisting of 41 proteins was identified (Table 5). Existence of a significant cluster was repeatedly shown to be associated with underlying disease biology. The observed response module not only uncovers the underlying biology of response but also served as Features set 2. In particular, FIG. 6 illustrates a classifier development flowchart containing identifying features of the classifier (A), training and validation of a probabilistic neural network on cohort 1 using identified features (B) and validation of the trained classifier using identified feature genes expressions in an independent cohort (C). The final set of features are selected based on best performance.

TABLE 5

| | #Top genes | #Genes | | |
|---|---|---|---|---|
| Cohort ID | selected | in HI | LCC size | Significance |
| 1 | 200 | 186 | 41 | 1.19 |

### Training the Response Classifier and In-Cohort Validation

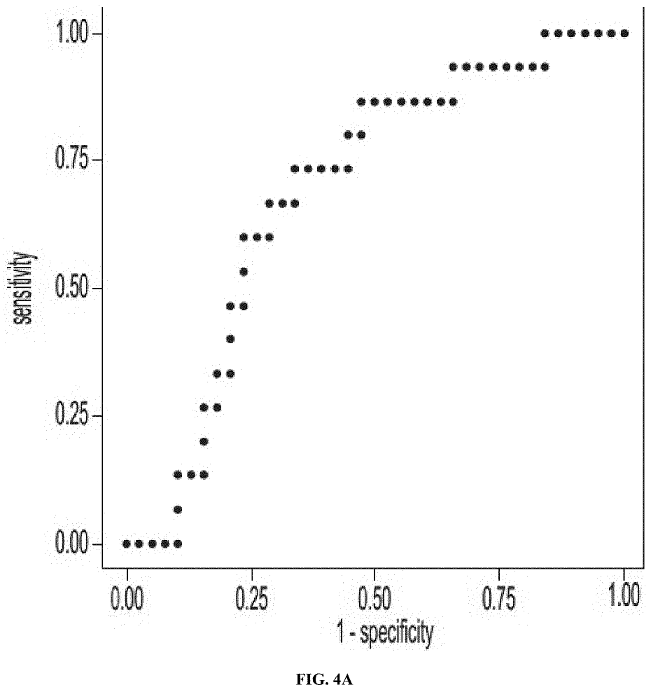

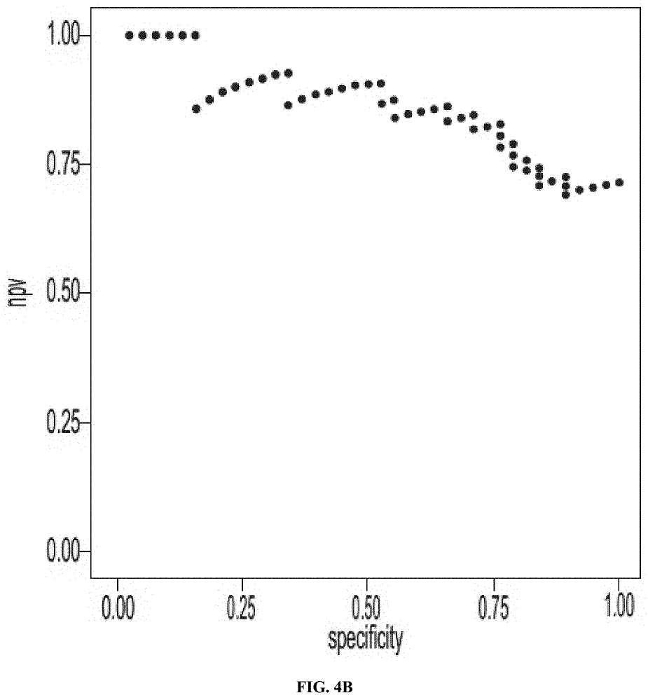

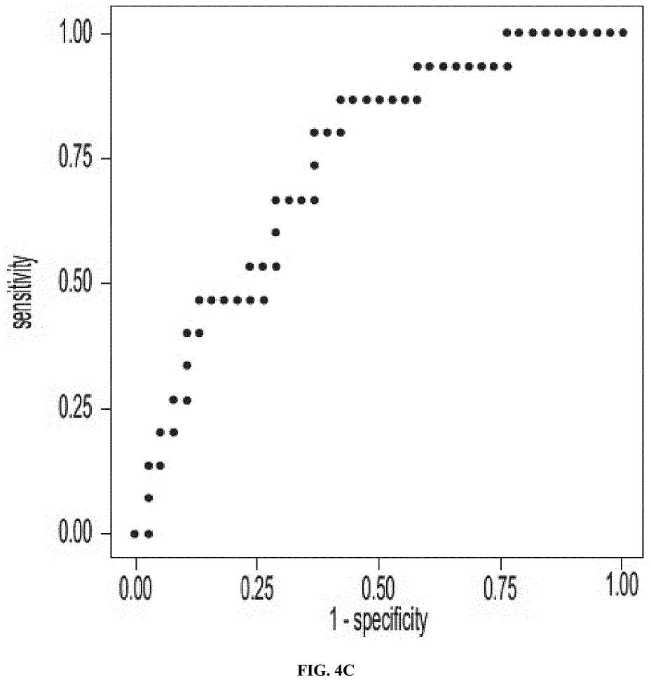

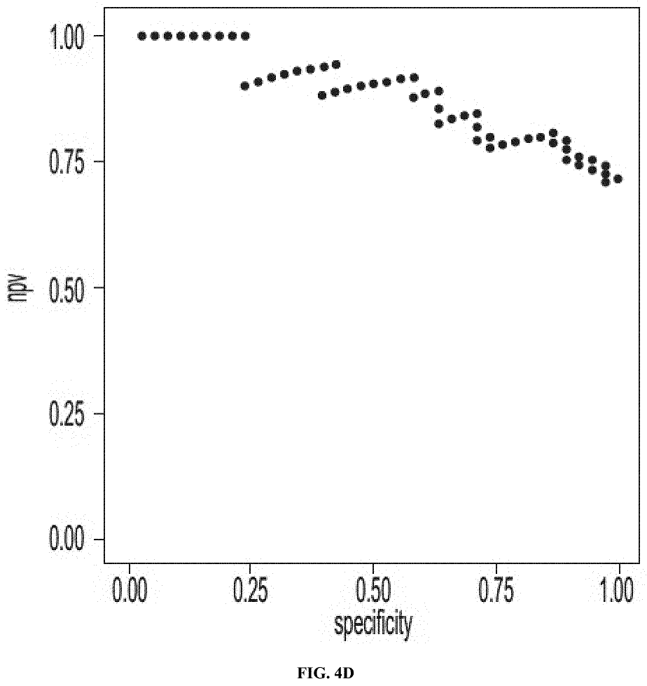

In the present Example, a response classifier was trained by feeding a probabilistic neural network with Feature set 1 and 2. Training the classifier on Feature set 1 significantly predicted response using leave-one-out cross validation and reached an AUC of 0.69, an NPV of 0.9 and a TNR of 0.52 (FIG. 4A, and FIG. 4B, respectively), outperforming Feature set 2. Having a smaller number of classifier genes also opens up the opportunity to use a variety of lower cost, FDA-approved expression platforms with a broad installed base to generate the required gene expression data sets. The classifier was therefore further trained to see if performance holds up when reducing the number of genes in Feature set 1 by training on top n-ranked genes where n goes from 1 to 20. A local maximum was observed in classifier performance when training on the top 9 genes (AUC=0.74, corrected p-value=0.006) with an NPV of 0.92 and a TNR of 0.76 (FIG. 4C and FIG. 4D). The 9-gene classifier was chosen for the cross cohort validation analysis below.

### Validation of Trained Response Classifier in an Independent Cohort (Cross-Cohort Validation)

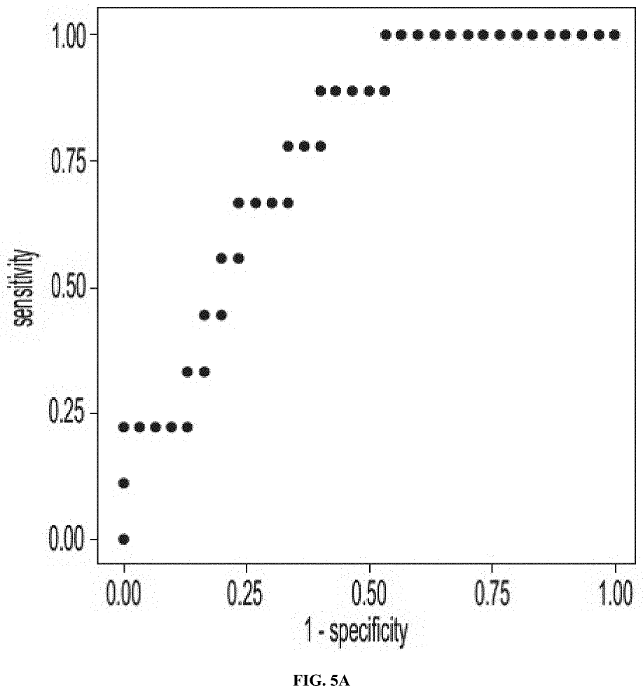

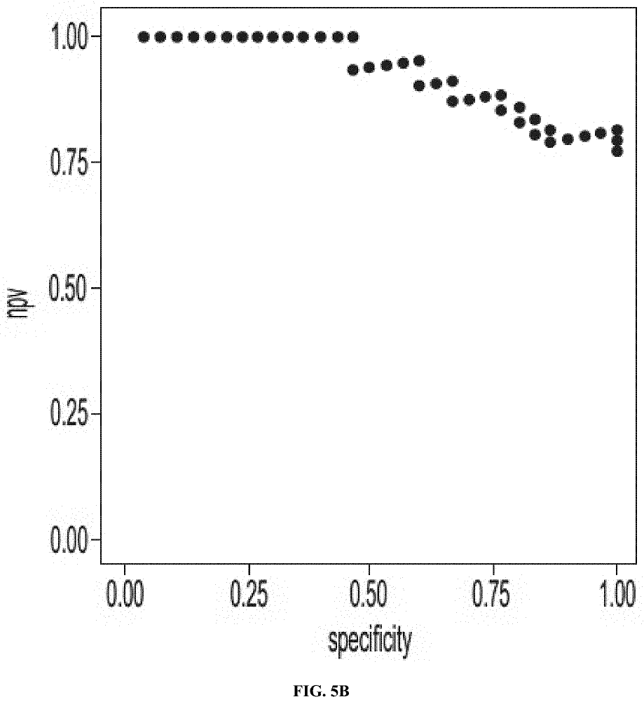

Of critical importance when building diagnostic tests and classifiers is the ability to reproduce the results and successfully test the classifier's performance in an independent cohort. The developed 9-gene classifier was therefore tested in a blinded fashion on a completely independent group of patients (cohort 2). The results show that the classifier performed well (cross-cohort AUC=0.78, p value=0.01) with an NPV of 0.91 and a TNR of 0.67 (FIG. 5B and Table 6). FIG. 5A is an ROC curve of cross-cohort classifier test results.

TABLE 6

| Predicting non-responders for TNF-naïve patients | AUC | NPV | TNR |
|---|---|---|---|
| Classifier trained on cohort 1 tested on cohort 2 | 0.78 | 0.91 | 0.67 |

### DISCUSSION

The present Example documents effectiveness of a classifier, as described herein, that predicts non-response to anti-TNF drugs before therapy is prescribed in patients suffering from RA.

Interviews with payers and clinicians indicate that current target specifications aim to identify at least half of the non-responders to anti-TNF therapy with high negative predictive accuracy (NPV>90%). Patients that are identified as non-responders can be placed on alternative effective therapies and higher response rates for those patients still offered anti-TNFs can be achieved. Financial savings are garnered by not spending on expensive ineffectual therapies and avoiding serious side effects and continuing disease progression. By identifying 50% of the non-responders, significant cost and care benefits can be achieved since, in the absence of stratification, two-thirds of patients do not achieve the target of LDA or remission today. High NPV is desired to ensure that few patients that would have responded are not incorrectly withheld a therapy they would have benefited from.

For RA, the present disclosure has demonstrated an AUC of 0.78, an NPV of 0.91 and a TNR of 0.67, resulting in the matrix below (Table 7). That is, the classifier identifies 67% of true non-responders with a 91% accuracy. Stratifying patients using this classifier would increase the response rate for the anti-TNF treated group by 71% from 34% to 58%. By comparison, the highest cross-cohort performance reported for classifiers developed by others had an NPV of 0.71 and a TNR of 0.71. See Toonen E J. et al. “Validation study of existing gene expression signatures for anti-TNF treatment in patients with rheumatoid arthritis.”PLoS One. 2012; 7(3):e33199. Using that classifier would significantly misclassify the genuine responders leading to a worse overall response rate than not using it at all. The presently described classifiers clearly meet the performance targets when tested in an independent cohort of patients.

TABLE 7

Predicted

| Actual | R | NR | | |
|---|---|---|---|---|
| R | 30 | 4 | 34 | TPR 87% |
| NR | 22 | 44 | 66 | TNR 67% |
| | 52 | 48 | | |
| | PPV 58% | NPV 91% | | |

The reduced number of genes in the classifier allows several expression analysis platforms to be considered for the delivery of the final commercial version of the test. For example, Nanostring nCounter system uses digital barcode technology to count nucleic acid analytes for panels of up to several hundred genes on an FDA approved platform. Multiplexed qRT-PCR is the gold standard for quantifying gene expression for panels of less than ˜20 genes and would enable the test to be offered as a distributable kit. RA is a chronic, complex auto-immune diseases, where many genetic risk factors have been identified but none of them are of sufficient impact to be useful as diagnostic or prognostic markers. The present disclosure provides a ranked list of candidate genes based on correlation of baseline expression level with response outcomes. The rank order is derived from the significance of the correlation. The present disclosure, however, does not prioritize genes with larger fold change across the category of responders and non-responders. It is common practice in the field to give preference to genes that show the highest fold change. This is because it is generally believed that large changes in expression levels are biologically more meaningful, and because of the technical advantage of high signal to noise ratios to compensate for high background and other sources of technical variability. However, the present disclosure appreciates that small differences, which are ignored or overlooked in many conventional technologies, can provide important, and even critical, discriminating capability. Without wishing to be bound by any particular theory, the present disclosure proposes that subtle differential perturbations may be particularly relevant and/or important in situations, like the present, where subjects suffering from the same disease, disorder, or condition are compared with one another (e.g., rather than with “control” subjects not suffering from the disease, disorder, or condition). It may be that small yet statistically significant differences in gene expression differentiate patient populations in complex diseases such as RA. This study shows that even very small but significant changes in gene expression will lead to a different treatment outcome. This method captures genes that are overlooked by conventional differential expression analysis.

Additionally, the present disclosure utilizes the highly unbiased and independently validated map of the protein-protein interactions in cells, the human interactome. By mapping the prioritized genes to the interactome, distinct and statistically significant clusters appear. In addition to using the interactome network analysis to define the classifier, the identified clusters also provide biological insights into the biology and causal genes of anti-TNF response. The genes corresponding to the top 9 genes in RA are valuable in immunological pathways and functions linked to ER stress, the protein quality control pathway, control of the cell cycle and the ubiquitin proteasome system, primarily in targeting key regulators of the cell cycle to the proteasome through ubiquitinyation.

The classifiers described here serve as the basis for diagnostic tests to predict anti-TNF non-response for patients with moderate to severe disease and considering initiating biologic therapy. Patients identified as non-responders will be offered alternative, approved mechanism of action therapies. These tests will provide significant improvements to current clinical practice by increasing the proportion of patients reaching treatment goals, making the treatment assignment based on scientific data and as a result decrease waste of resources and generate significant financial savings within the health care system.

### Materials and Methods

RA Cohort Description and Microarray Analysis

Blood samples were collected from RA patients across the United States from two individual observational studies, both of which predominantly consisted of older Caucasian women. Cohort 1 was obtained from a multi-center study conducted in 2014. These patients were treated with Enbrel, Remicade, Humira, Cimzia and Simponi. Cohort 2 was obtained from the Autoimmune Biomarkers Collaborative Network, a NIAMS supported contract to develop new approaches to biomarkers for RA and lupus in 2003. These patients were treated with Humira, Remicade and Enbrel.

The level of response was defined using the EULAR DAS28 scoring criteria assessed 14 weeks after anti-TNF treatment. EULAR response rates for female TNF naïve patients are given in Table 1. EULAR response characterizes patients into good responders, moderate responders and non-responders. For this study, response was defined as EULAR good response, or DAS28 improvement>1.2. This corresponds to LDA or remission.

The gene expression data and 14 week response outcome was available for 50 and 39 female and TNF naïve samples in cohort 1 and 2, respectively, for classifier design and validation.

All subjects had PaxGene tubes drawn at baseline before starting therapy, and again at 14 weeks after treatment started. RNA was isolated using the QIAcube (Qiagen) following the manufacturer's automated protocol for PaxGene blood RNA. Extracted samples were eluted in 80 ul of elution buffer (BR5) and subsequently run on Agilent's 2100 Bioanalyzer of RNA integrity using the RNA 6000 Nanochip. Samples with RNA Integrity Numbers (RIN)>6.5 were diluted to 30 ng/μl in a total 11 μl of RNAse-free water. Samples were amplified using Life Technologies Illumina® RNA Total Prep Amplification Kit. 750 ng of cRNA was re-suspended in 5 μl of RNAse-free water for analysis on the Illumina® Human HT-1.2v4 chip (cohort 1 samples) and 1.2 μg was re-suspended in 10 μl of RNAse-free water for analysis Illumina® WG6v3 Bead Chip (cohort 2 samples). All samples were processed according to the manufacturer's instructions.

Raw data were exported from GenomeStudio® and further analyzed with the R programming language. All datasets were background corrected using the R/Bioconductor package “lumi.” Data were further transformed using variance stabilization transformational (vst) and quantile normalized. Probes with zero detection count and detection rates of lower that 50% across samples were removed from the study. To enable cross cohort classifier testing, the two cohorts were combined and normalized using the ComBat package in R and then separated to ensure completely blind testing. All of the microarray analysis resulted in having about 10,000 common probes in the two cohorts.

Identification of Classifier Genes

Genes with expression values that are significantly correlated to clinical measures after treatment are selected as the best determinants of response. Expression correlation of gene expression to response outcome is measured by Pearson correlation. Genes are ranked based on the correlation value and the performance of the classifier is assessed when using top n ranked genes. In some cases mapping the ranked genes on the interactome forms a significant cluster reflecting the underlying biology of response. It is observed that the ranked genes are not randomly scattered on the network. Instead, they significantly interact with each other, reflecting the existence of an underlying disease biology module that explains response.

Classifier Design and Validation

Genes identified in the previous step were used as features of a probabilistic neural network. In this approach the average distance of each sample to training samples' probability distribution functions is calculated. The average distance of a test sample to training samples in the n-dimensional feature space determines the probability of belonging to one group vs. the other. The classifier was validated using leave-one-out cross validation within a given cohort. In this approach, the classifier was trained based on the outcome data provided on all patients but the one left out. The classifier was blind to the response outcome of that left out patient. Predicting the outcome of the patient that has been left out then validated the trained classifier. This procedure was repeated so that each patient was left out once. The classifier provided a probability for each patient reflecting whether they responded or not. These probabilities were used to define a score (by using log of likelihood ratio) for each patient. The area under the curve (AUC) determined the performance of the classifier. In cross-cohort assessment of the classifier, the trained classifier was completely blind to the outcome of the independent cohort. Trained data on one cohort is tested to determine its ability to predict response in an independent cohort.

Statistical Analysis

Fisher's t-test was used to determine the significance of difference between two distributions.

Human Interactome

The human interactome contains experimentally supported physical interactions between cellular components. These interactions are collected from several resources but only those supported by a rigorous experimental validation confirming the existence of a physical interaction between proteins are selected. Most of the interactions in the interactome are from unbiased high-throughput studies such as yeast 2-hybrid. Experimentally supported interactions that that have been reported in at least two publications are also included. These interactions include regulatory, metabolic, signaling and binary interactions. The interactome contains about 17,000 cellular components and over 200,000 interactions. Unlike other interaction databases the present methods do not include any computationally inferred interactions, nor any interaction curated from text parsing of literature with no experimental validation. Therefore, the interactome used is the most complete, carefully selected and quality controlled version to date.

### Example 3: Determining a Gene Expression Response Signature—Ulcerative Colitis

The present examples provide a network-based response module comprised of gene expression biomarkers that predict response or non-response to an anti-TNF therapy (also referred to as TNF inhibitors, or, “TNFi” or “TNFis”, including infliximab) at treatment initiation in ulcerative colitis.

### Cohort Description

In the present example, two cohorts were studied. Cohort A (GSE14580) included twenty-four patients with active ulcerative colitis (UC), refractory to corticosteroids and/or immunosuppression, and underwent colonoscopy with biopsies from diseased colon within a week prior to the first intravenous infusion of 5 mg infliximab per kg body weight. Response to infliximab was defined as endoscopic and histologic healing at 4-6 weeks after first infliximab treatment. Eight patients were determined to be responders, sixteen were determined to be non-responsive. Six control patients with normal colonoscopy were included. Total RNA was isolated from colonic mucosal biopsies, labelled, and hybridized to Affymetrix® Human Genome U133 Plus 2.0 Arrays.

Cohort B (GSE12251) included twenty-two patients who underwent colonoscopy with biopsy before infliximab treatment. Response to infliximab was defined as endoscopic and histologic healing at week 8 (12 patients as responders and 11 patients as non-responders). Messenger RNA was isolated from pre-infliximab biopsies, labeled and hybridized to Affymetrix® Human Genome U133 Plus 2.0 Array.

### Microarray Analysis

The two datasets were downloaded using GEOquery R package. Before treatment gene expression data were extracted by setting the visit time point to baseline. Probe IDs were converted to gene Entrez ID using the hgu133plus2.db database. The two datasets were merged by the common probe IDs. Batch effects were removed using ComBat from the sva R package. To retain the biological differences between responders and non-responders, cohort-specific biomarkers were derived prior to applying ComBat.

### Human Interactome

The Human Interactome, previously described in Menche et al., Science, 347(6224): 1257601 (Feb. 20, 2015), contains experimentally determined physical interactions between proteins. These interactions include, regulatory, metabolic, signaling, and binary interactions. The Human Interactome amalgamates data from more than 300 thousand interactions among them.

### Identification of Classifier Genes (i.e., Genes of the Gene Expression Response Signature)

For all genes in each cohort, Pearson correlation between their gene expression values and response to treatment was determined. The signal-to-noise ratio of each gene correlation was calculated by randomly shuffling of the response outcome 100 times. Selected genes were then mapped onto the consolidated Human Interactome, and the largest connected component (LCC), was determined.

### Classifier Design and Validation

Genes identified as discriminatory between responders and non-responders to infliximab that were in the LCC were used as features of a probabilistic neural network. Gonzalez-Camacho, et al., BMC Genomics. 17:208. (Mar. 9, 2016). One cohort was selected for classifier training using the R package pnn, while the second cohort was used for blinded independent validation. The in-cohort model training and validation was done using a leave-one-sample-out cross validation where the classifier was blind to the response outcome of that left-out patient. The classifiers were validated using leave-one-batch out cross-validation where one cohort was used for feature selection and model training and the other cohort was used for independent validation.

The classifier was trained using the default smoothing parameter (σ=0.8).

The classifier provided a probability for each patient reflecting whether or not that individual responded to infliximab. The log likelihood ratio of response and non-response probabilities were used to define a score for each patient and draw the receiver operating characteristic (ROC) curves by comparing the score to actual response outcomes. The area under the curve (AUC) determined the performance of the classifiers. In cross-cohort assessment of classifiers, the trained classifiers were blind to the outcome of the independent cohort.

### Response Module Statistics and Randomization

One of the shared genes (UBC) between the two top-200 gene sets was a high degree node in the Human Interactome, that could have caused a high degree of perceived connectedness between set of LCC genes from the two cohorts. To control and correct for the effect of the high degree node and the many shared nodes between the gene sets, nodes were randomly assigned to one cohort while the shared genes were preserved between two sets during the randomization.

### Results

Identification of gene expression features predictive of non-response to infliximab

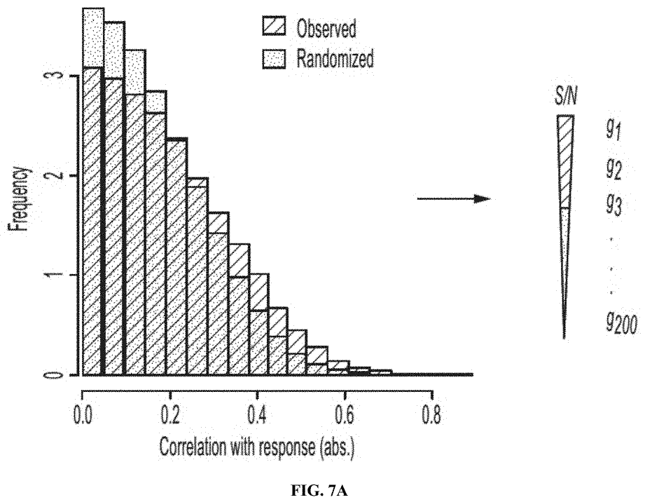

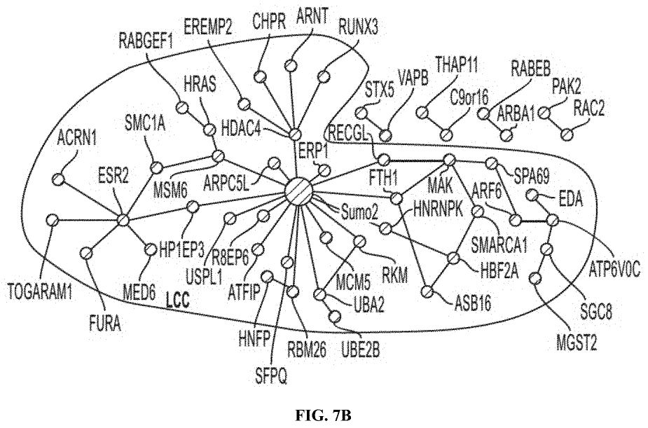

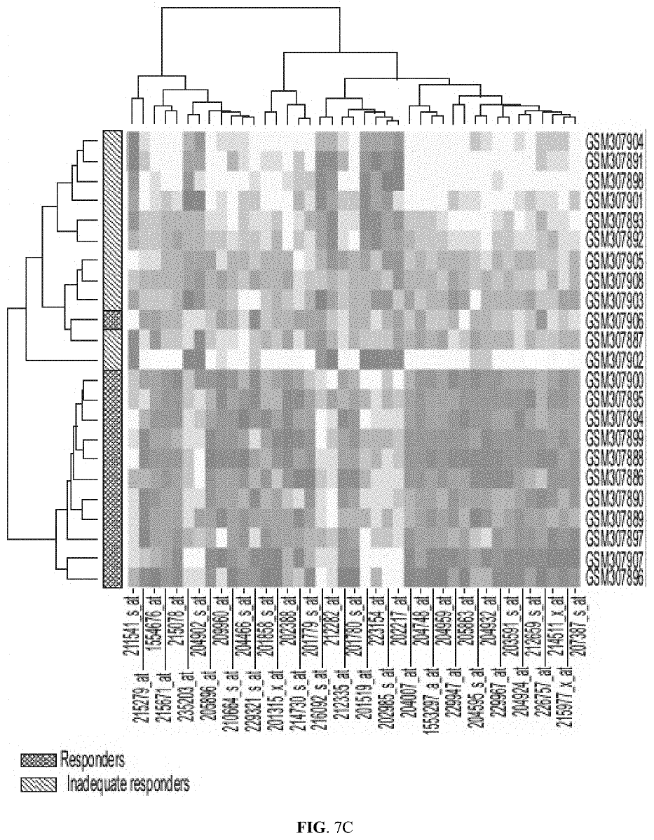

To identify genes whose expression best distinguishes responders from non-responders (also referred to as “inadequate responders”) to infliximab, two publicly available UC patient gene expression datasets were downloaded for which the clinical outcomes data were available. Arijs I, et al. Gut. 58(12):1612-9 (2009). Each cohort was separately analyzed to find genes with significant gene expression deviations between responders and inadequate responders. Santolini M, et al. NPJ Syst Biol Appl. 4:12 (2018). Unlike conventional differential expression methods that look for large fold-changes in gene expression between two groups, this analysis investigated small but significant changes—a high signal-to-noise ratio—between the two cohorts. Genes were ranked by decreasing value of signal-to-noise ratio and the top 200 genes with the highest signal-to-noise ratio were selected as infliximab response discriminatory genes (FIG. 7A).

Refinement of Molecular Signature Genes Using the Human Interactome

The Human Interactome network map of protein-protein interactions can serve as a blueprint to better understand the interconnectivity and underlying biology of the response prediction genes. The top 200 genes from each cohort whose expression values across patients were significantly correlated to clinical outcome after infliximab treatment were selected and mapped onto the Human Interactome (FIGS. 7B-7C). Although these genes were identified from gene expression data only, the proteins encoded by these genes formed a significant cluster on the Human Interactome, with 182 and 193 proteins for Cohort A and B, respectively. The LCC on the Human Interactome for each set of response prediction genes was larger than expected by chance; the cohort A LLC was 39 genes (z-score of 2.91) and the cohort B LCC was 41 genes (z-score of 2.33). Menche J, et al. Science. 347(6224):1257601 (2015); Sharma A, et al. Hum Mol Genet. 24(11):3005-20 (2015); Barabasi A L, et al. Nat Rev Genet. 12(1):56-68 (2011); Ghiassian S D, et al. Sci Rep. 6:27414 (2016). Z-scores>1.6 have been associated with sub-networks of underlying disease biology. Among the lists of LCC genes, two genes (PKM and SUMO2) were in common between the two cohorts. See Table 8, below.

TABLE 8

| | |
|---|---|
| Cohort A | Cohort B |

| | | | |
|---|---|---|---|
| ADAR | NUCKS1 | ARCN1 | MCM5 |
| ANP32B | PKM | ARF6 | MED6 |
| ATRX | PML | ARNT | MGST2 |
| BRD7 | PNN | ARPC5L | MSH6 |
| CAPN1 | PRKAB1 | ASB16 | PKM |
| CCDC88A | RBCK1 | ATF7IP | PURA |
| CFAP206 | RRP15 | ATP6V0C | RABGEF1 |
| CGN | SNRPN | BRF1 | RBBP6 |
| CIRBP | SUMO2 | CHFR | RBM26 |
| CLTC | TFIP11 | EDA | RECQL |
| EEA1 | THTPA | EFEMP2 | RUNX3 |
| ERICH1 | TMEM87A | ESR2 | SFPQ |
| FAM192A | TNK2 | FAM179B | SGCB |
| FAM207A | TPR | FTH1 | SMARCA1 |
| HHEX | TRAPPC4 | H3F3A | SMC1A |
| KLF3 | UBA5 | HDAC4 | SPAG9 |
| LCA5 | UBE2D1 | HINFP | SUMO2 |
| MDC1 | VPS72 | HNRNPK | UBA2 |
| MDM2 | YWHAE | HP1BP3 | UBE2B |
| NFAT5 | | HRAS | USPL1 |
| | | MAX | |

Classifier Training and Blinded Cross-Cohort Validation

For each cohort, the LCC genes were used to train a probabilistic neural network. See Specht D F. IEEE Transactions on Electronic Computers. EC-16(3):308-19 (1967); Specht D F. IEEE Trans Neural Netw. 1(1):111-21 (1990). A probabilistic neural network is an optimum pattern classifier that minimizes the risk of incorrectly classifying an object with high efficiency. Gonzalez-Camacho J M, et al., BMC Genomics. 17:208 (2016). For each cohort, the probabilistic neural networks were trained using the LCC genes and patient data to teach the predictive classifiers the appropriate patient outcome (i.e., response or inadequate response to infliximab) for each input (i.e. gene expression levels of LCC genes).

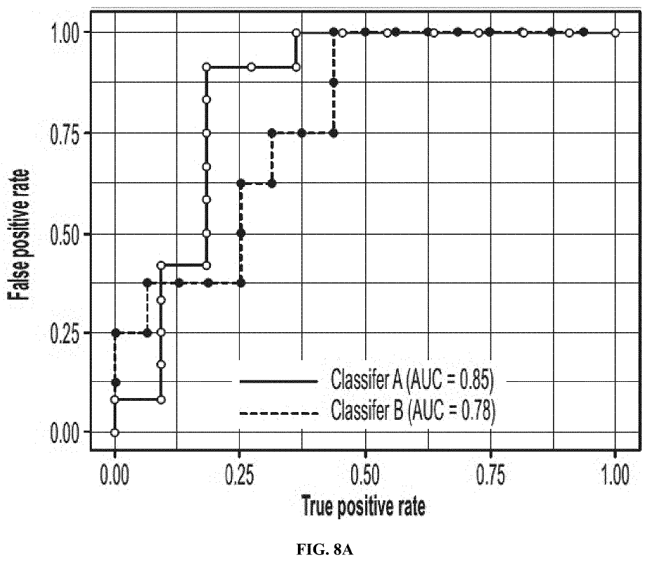

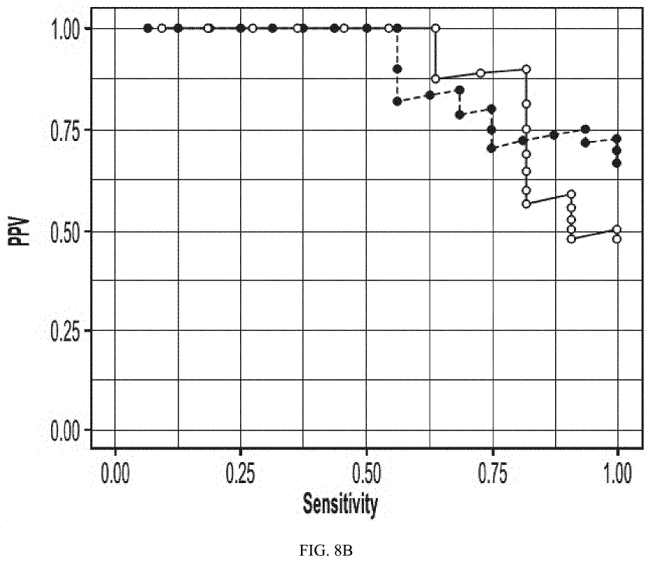

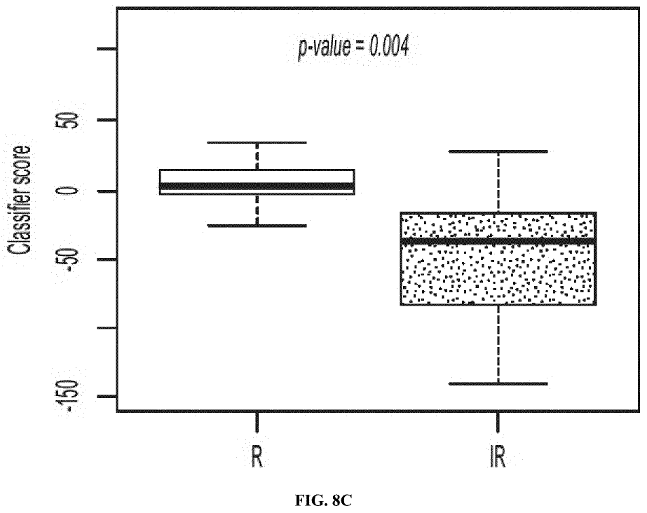

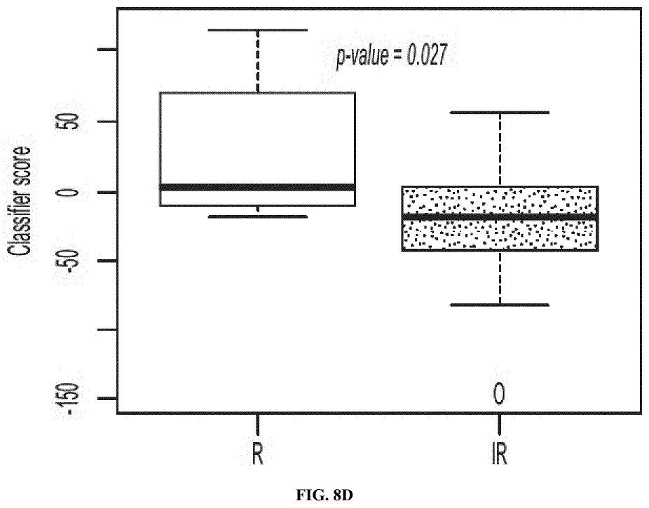

Blinded, independent cross-cohort validation assessed the performance of the two predictive classifiers. In this analysis, the classifier that was trained on the known data and outcomes from one cohort was used to predict the outcomes on the other cohort, ultimately testing the ability of the predictive classifiers to accurately predict inadequate response to infliximab in an unseen patient population. To assess the performance of the classifiers, the classifier predicted probabilities were converted to a continuous classifier prediction score using log-likelihood ratio. ROC curves, which plot the rate of false positives versus the rate of true positives, were used to assess cross-cohort performance (FIG. 8A). Although the two LCCs had only two genes in common, the predictive classifiers showed significantly high performance. An AUC of 0.85 was observed for classifier trained on cohort A predicting response to infliximab among cohort B patients and an AUC of 0.78 was observed for classifier trained on cohort B predicting response to infliximab among cohort A patients. Additionally, the cross-cohort positive predictive value (“PPV”, which has been referred to previously as “negative predictive value” or “NPV” in earlier examples and in work by others) and sensitivity (also referred to previously and by others as “specificity”) were estimated (FIG. 8B), which are metrics that describe the accuracy of the inadequate response predictions. At a 90% PPV, classifier A had a sensitivity of 82% and classifier B had a sensitivity of 56%. The distribution of classifier prediction scores in responders and inadequate responders when validated in independent cohorts showed a significant difference between the classifier prediction scores for responders and inadequate responders (FIGS. 8C-8D).

The UC infliximab response module is a sub-network on the Human Interactome

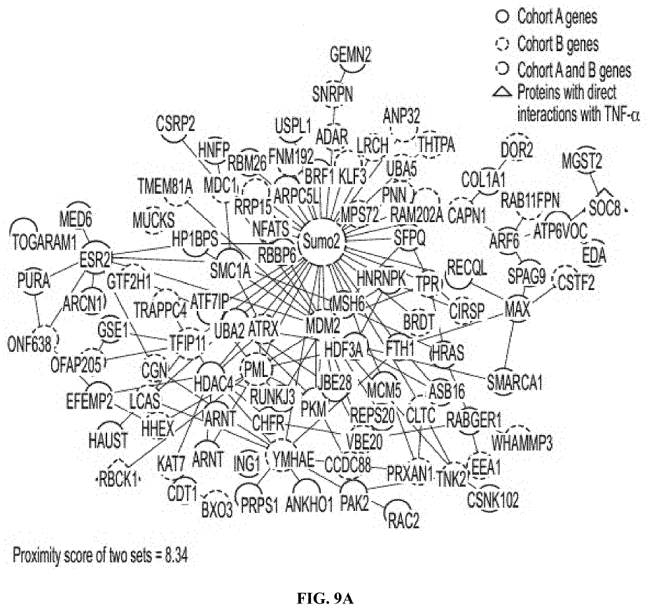

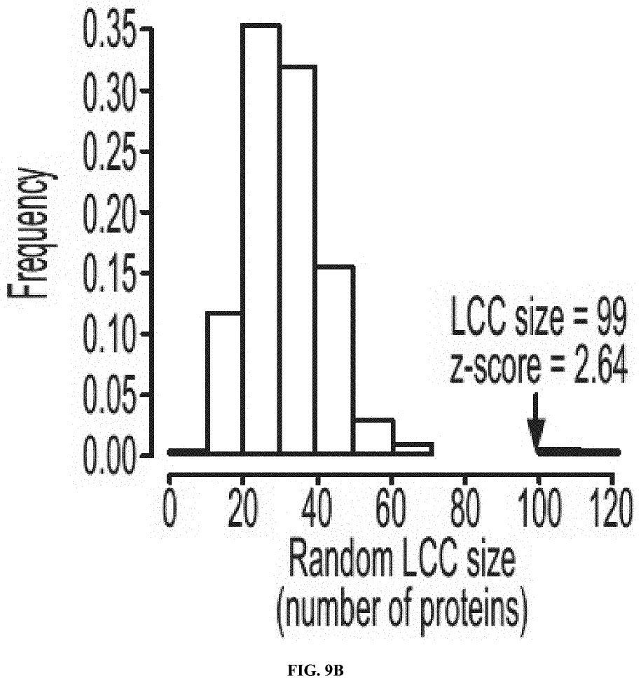

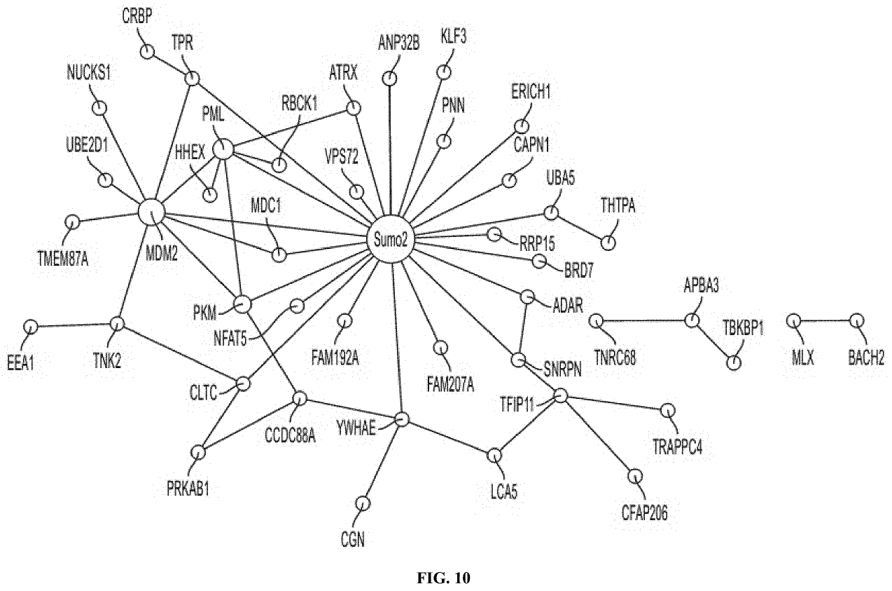

The high cross-cohort performance, despite the limited overlap between LCC gene sets, motivated the search for an underlying mechanism that explained the biology of inadequate response to infliximab in UC patients. When the 200 top genes from the two cohorts were mapped simultaneously onto Human Interactome, the genes were not randomly scattered on the network, but instead significantly interacted with each other (z-score of 8.34) forming a common LCC (FIG. 9B) that was significantly larger than the random expectation (99 genes; z-score of 2.64). To account for genes that were shared between the two cohort gene lists, including a high-degree node (UBC) on the Human Interactome, a careful randomization was made to estimate the significance of interconnectivity. Two proteins in the common LCC (RBCK1 and SGCB) are direct interaction partners of TNF-α, the protein target of infliximab. Several proteins in the common LCC were orphan genes that were not previously part of LCCs of the individual cohorts (e.g. GEMIN2 and CSTF2) yet were integrated into this common LCC (FIG. 9A). Our results show that even though the biomarkers identified from each cohort were apparently distinct with minimal overlap, their protein products tend to interact significantly on the network, reflecting the existence of an underlying disease biology sub-network, or response module, that defines a molecular signature of inadequate response to infliximab in UC patients.

### DISCUSSION

This present example describes two predictive classifiers developed using knowledge from the Human Interactome map of protein-protein interactions and a probabilistic neural network machine learning algorithm. The genes predictive of response to infliximab identified from baseline colon biopsy samples from two separate patient cohorts showed limited overlap in identity but significant overlap on the Human Interactome and were predictive of response to infliximab in a cross-cohort validation. The patients in these two cohorts are all diagnosed with UC, and as such, differences in the biology between these individuals may not manifest in large fold-changes in gene expression. These subtle differences in transcript levels may be overlooked in conventional differential gene expression analyses. However, this study identified small but significant changes in gene expression that may lead to different treatment outcomes.

There is an interaction between genetic, immune, and environmental factors that is evident in the mucosa gene expression profiles of IBD patients compared to healthy controls and in the genetic risk alleles associated with an increased risk of IBD. Jostins L, et al. Nature. 491(7422):119-24 (2012). The topological and biological properties of the infliximab response module on the Human Interactome suggests that it is possible to determine a molecular signature for inadequate response to TNFi therapies in patients with UC. TNFi therapies have demonstrated efficacy in the treatment of moderate to severe IBD. However, response rates vary, and initially 40-60% of patients fail to achieve remission with their initial treatment, dose escalation is needed in 23-46% of patients after 12 weeks of treatment and up to 50% of patients who responded initially will have a secondary loss of response after 12 months of therapy. Ford A C, et al. Am J Gastroenterol. 106(4):644-59, quiz 60 (2011); Sandborn W J, et al. Gastroenterology. 142(2):257-65 el-3 (2012); Zampeli E, et al. World J Gastrointest Pathophysiol. 5(3):293-303 (2014); Rutgeerts P, et al. N Engl J Med. 353(23):2462-76 (2005); Roda G, et al. Clin Transl Gastroenterol. 7:e135 (2016); Fausel R, Afzali A. Ther Clin Risk Manag. 11:63-73 (2015); Fine S, et al., Gastroenterol Hepatol (N Y). 15(12):656-65 (2019).

Given the need to rapidly manage disease flares and avoid surgery, there is a critical need for a test that can predict which UC patients will benefit from TNFi therapy and who should consider alternative treatment options.

The two sets of response prediction genes described in this study have little overlap; however, they are unified in a common response module on the Human Interactome. This observation addresses one of the major concerns of biomarker irreproducibility; studies evaluating response prediction biomarkers rarely report the same genes. Many studies have reported prognostic indicators of response to TNFi therapies in UC. Arijs I, et al. Gut. 58(12):1612-9 (2009); Subramaniam K, et al. Intern Med J 44(5):464-70 (2014); Garcia-Bosch O, et al. J Crohns Colitis. 7(9):717-22 (2013); Rismo R, et al. Scand J Gastroenterol. 47(5):538-47 (2012); Olsen T, et al. Cytokine. 46(2):222-7 (2009).

A gene array study of UC mucosal biopsies identified gene panels predictive of response to infliximab with 95% sensitivity and 85% specificity. Arijs I, et al. Gut. 58(12):1612-9 (2009). A prospective study determined the predictive value of pre-treatment mucosal T cell-related cytokine gene expression profiles in response to infliximab; expression of transcripts encoding IL-17A and IFN-γ were associated with remission after three infliximab infusions (OR=5.4, p=0.013 and OR=5.5, p=0.011, respectively). Rismo R, et al. Scand J Gastroenterol. 47(5):538-47 (2012). These studies developed predictive models using machine learning approaches, calculating mean gene expression values, evaluating the highest fold changes in gene expression and/or taking a pathway-based approach to describe UC disease biology. None of these studies have been developed into a clinical test for care of UC patients. By mapping the response module, network analyses performed in this study enabled identification of biomarkers associated with a specific disease phenotype (inadequate response to infliximab), reduced the noise inherent to gene expression data and eliminated many false positives that can arise from small sample sizes and characteristics specific to demographics of a particular patient cohort.

This network-based approach evaluates protein interactions to select genes that reflect the biology of disease at the individual patient level. The cross-cohort validation of two predictive classifiers, developed using a response module found in the Human Interactome, suggests the existence of a molecular signature in baseline tissue samples that characterizes UC patients who will have an inadequate response to TNFi therapy. Further development of such a test would decrease the time to treatment response, thus allowing patients to get back to their normal, productive lives sooner while decreasing the burden on supportive family members. Furthermore, this method of biomarker discovery and classifier development can be applied across multiple disease areas with complex phenotypes and datasets containing molecular information. The platform described herein opens new, unprecedented opportunities to create new drug response modules, predict drug response in complex diseases, and achieve the goal of treating patients with the most effective treatment for their unique disease biology.

The foregoing has been a description of certain non-limiting embodiments of the subject matter described within. Accordingly, it is to be understood that the embodiments described in this specification are merely illustrative of the subject matter reported within. Reference to details of the illustrated embodiments is not intended to limit the scope of the claims, which themselves recite those features regarded as essential.

It is contemplated that systems and methods of the claimed subject matter encompass variations and adaptations developed using information from the embodiments described within. Adaptation, modification, or both, of the systems and methods described within may be performed by those of ordinary skill in the relevant art.

Throughout the description, where systems are described as having, including, or comprising specific components, or where methods are described as having, including, or comprising specific steps, it is contemplated that, additionally, there are systems encompassed by the present subject matter that consist essentially of, or consist of, the recited components, and that there are methods encompassed by the present subject matter that consist essentially of, or consist of, the recited processing steps.

It should be understood that the order of steps or order for performing certain action is immaterial so long as any embodiment of the subject matter described within remains operable. Moreover, two or more steps or actions may be conducted simultaneously.

1-47. (canceled)

48. A method of treating a subject suffering from ulcerative colitis (UC), the method comprising:
administering to the subject an anti-TNF therapy,
wherein the subject has been predicted to be responsive to the anti-TNF therapy based at least in part on a trained machine learning classifier that distinguishes between responsive and non-responsive subjects who have received the anti-TNF therapy, and
wherein the trained machine learning classifier distinguishes between responsive and non-responsive subjects, based at least in part on analyzing an expression level in the subject of a set of genes.

49. The method of claim 48, wherein the trained machine learning classifier further analyzes:
presence of one or more single nucleotide polymorphisms (SNPs) in a sequence of one or more genes that is expressed in the subject; or
presence of one or more clinical characteristics of the subject.

50. The method of claim 49, wherein the one or more clinical characteristics of the subject comprise body-mass index (BMI), gender, age, race, previous anti-TNF therapy treatment, disease duration of ulcerative colitis (UC), C-reactive protein level, or treatment response rate to the anti-TNF therapy.

51. The method of claim 48, wherein the trained machine learning classifier predicts the subject to be responsive to the anti-TNF therapy using a non-linear relationship between (i) an expression level of one or more genes identified in the subject and (ii) responsiveness or non-responsiveness to the anti-TNF therapy.

52. The method of claim 48, wherein the trained machine learning classifier is trained using expression levels of a set of genes in (i) a first set of subjects with ulcerative colitis (UC) who were responsive to the anti-TNF therapy and (ii) a second set of subjects with ulcerative colitis (UC) who were non-responsive to the anti-TNF therapy.

53. The method of claim 52, wherein the trained machine learning classifier is validated by validating the classifier on a second independent cohort of subjects who have received the anti-TNF therapy and have been determined as either responding to the anti-TNF therapy or not responding to the anti-TNF therapy.

54. The method of claim 53, wherein validating the classifier further comprises using the classifier to predict a probability of response of at least one of the second independent cohort of subjects.

55. The method of claim 48, wherein the trained machine learning classifier comprises a neural network or a random forest.

56. The method of claim 48, wherein the trained machine learning classifier predicts that subjects within a population are responsive or non-responsive to the anti-TNF therapy with a true negative rate (TNR) of at least about 60%.

57. The method of claim 48, wherein the trained machine learning classifier predicts that subjects within a population are responsive or non-responsive to the anti-TNF therapy with a negative predictive value (NPV) of at least about 85%.

58. The method of claim 48, wherein the trained machine learning classifier predicts that subjects within a population are responsive or non-responsive to the anti-TNF therapy with an area under the curve (AUC) of at least about 70%.

59. The method of claim 48, wherein the trained machine learning classifier predicts that subjects within a population are responsive or non-responsive to the anti-TNF therapy with an accuracy of at least about 90%.

60. The method of claim 48, wherein the expression level is obtained by microarray, RNA sequencing, real-time quantitative reverse transcription PCR (qRT-PCR), bead array, or ELISA.

61. The method of claim 48, wherein the set of genes comprises: PKM, ADAR, ANP32B, ATRX, BRD7, CAPN1, CCDC88A, CFAP206, CGN, CIRBP, CLTC, EEA1, ERICH1, FAM192A, FAM207A, HHEX, KLF3, LCA5, MDC1, MDM2, NFAT5, ARCN1, ARF6, ARNT, ARPCSL, ASB16, ATF7IP, ATP6VOC, BRF1, CHFR, EDA, EFEMP2, ESR2, FAM179B, FTH1, H3F3A, HDAC4, HINFP, HNRNPK, SUMO2, NUCKS1, PML, PNN, PRKAB1, RBCK1, RRP15, SNRPN, TFIP11, THTPA, TMEM87A, TNK2, TPR, TRAPPC4, UBA5, UBE2D1, VPS72, YWHAE, MCM5, MED6, MGST2, MSH6, PURA, RABGEF1, RBBP6, RBM26, RECQL, RUNX3, SFPQ, SGCB, SMARCA1, SMC1A, SPAG9, UBA2, UBE2B, USPL1, HP1BP3, HRAS, or MAX.

62. The method of claim 48, wherein the set of genes comprises: SUMO2, ADAR, ANP32B, ATRX, BRD7, CAPN1, CCDC88A, CFAP206, CGN, CIRBP, CLTC, EEA1, ERICH1, FAM192A, FAM207A, HHEX, KLF3, LCA5, MDC1, MDM2, NFAT5, PKM, NUCKS1, PML, PNN, PRKAB1, RBCK1, RRP15, SNRPN, TFIP11, THTPA, TMEM87A, TNK2, TPR, TRAPPC4, UBA5, UBE2D1, VPS72, or YWHAE.

63. The method of claim 48, wherein the set of genes comprises: SUMO2, ARCN1, ARF6, ARNT, ARPC5L, ASB16, ATF7IP, ATP6VOC, BRF1, CHFR, EDA, EFEMP2, ESR2, FAM179B, FTH1, H3F3A, HDAC4, HINFP, HNRNPK, HP1BP3, HRAS, MAX, PKM, MCM5, MED6, MGST2, MSH6, PURA, RABGEF1, RBBP6, RBM26, RECQL, RUNX3, SFPQ, SGCB, SMARCA1, SMC1A, SPAG9, UBA2, UBE2B, USPL1.

64. The method of claim 48, wherein the set of genes comprises: SUMO2 and PKM.

65. The method of claim 48, wherein the anti-TNF therapy comprises: infliximab, adalimumab, etanercept, certolizumab pegol, golimumab, or a biosimilar thereof.

66. The method of claim 48, wherein an alternative to the anti-TNF therapy is administered when the trained machine learning classifier predicts the subject to be non-responsive to the anti-TNF therapy.

67. The method of claim 66, wherein the alternative to the anti-TNF therapy comprises: rituximab, sarilumab, tofacitinib citrate, leflunomide, vedolizumab, tocilizumab, anakinra, abatacept, or a biosimilar thereof.
